(19) United States
(12) Patent Application Publication
(10) Pub. No.: US 2010/0144737 A1
(43) Pub. Date: Jun. 10, 2010

(54) METHODS FOR INHIBITING T HELPER CELL DIFFERENTIATION

(75) Inventors: Felice Aisha Chow, San Francisco, CA (US); Stephen J. Klaus, San Francisco, CA (US); Ingrid Langsetmo Parobok, San Francisco, CA (US)

Correspondence Address: FIBROGEN, INC., 409 Illinois Street, San Francisco, CA, 94158, US

(73) Assignee: FibroGen, Inc., San Francisco, CA (US)

(21) Appl. No.: 12/316,028

(22) Filed: Dec. 8, 2008

(51) Int. Cl. A61K 31/50; C12N 5/02; A61K 31/44; A61K 31/19

(52) U.S. Cl. 514/248; 435/377; 514/301; 514/574

(57) ABSTRACT

The present invention relates to methods and compounds useful for inhibiting T helper cell differentiation. Methods and compounds for decreasing IL-12 signaling in T helper cells are also provided.

### FIELD OF THE INVENTION

The present invention relates to methods and compounds useful for inhibiting T helper cell differentiation. Methods and compounds for decreasing IL-12 signaling in T helper cells are also provided.

### BACKGROUND OF THE INVENTION

T helper (Th) cells are a sub-group of white blood cells (i.e., lymphocytes) that play an important role in establishing and maximizing the capabilities of the immune system. T helper cells are involved in activating and directing the activities and functions of other immune cells. For example, T helper cells are essential in determining B cell antibody class switching, in the activation and growth of cytotoxic T cells, and in maximizing bactericidal activity of phagocytic cells, such as macrophages.

As part of T cell activation, na\dot{i}ve T helper cells (i.e., Th0 cells) react to antigens presented on HLA class II molecules (antigen stimulation) and become either a Th1 or Th2 cell, a decision that is primarily influenced by cytokines within the local environment. If IL-12 is the major cytokine present during antigen stimulation, a Th1 response occurs and a Th2 response is inhibited; if IL4 is the major cytokine present during antigen stimulation, a Th2 response occurs and a Th1 response is inhibited. Therefore, IL-12 and IL4 play decisive roles in the differentiation of na\dot{i}ve T helper cells into Th1 cells or Th2 cells. Once activated, Th1 cells (also referred to as pro-inflammatory T cells) produce predominantly inflammatory cytokines, including IL-2, TNF-α, TNF-β, IL-1, and IFN-γ and are associated with cell-mediated immune responses; Th2 cells produce predominantly anti-inflammatory cytokines, including IL4, IL-5, IL-6, IL-10, and IL-13 and are associated with humoral immune responses. (Fitch et al. (1993) Ann Rev Immunol 11:2948.)

Pathological Th1 responses are associated with a number of organ-specific and systemic autoimmune conditions, including chronic inflammatory diseases and delayed type hypersensitivity reactions. Current methods used to reduce Th1 responses, including various non-specific immunosuppressive agents, such as, for example, cyclosporine and azathioprine, often require administration of therapeutic agents in high doses and are thus associated with toxicity and adverse side effects. In certain situations, it would be desirable to reduce cell-mediated immune responses (i.e., reduce Th1-mediated responses) without affecting humoral immune responses (i.e., Th2-mediated responses). Accordingly, there is need for novel methods that inhibit differentiation of T helper cells into Th1 cells differentiation and inhibit Th1 cell responses, such as those mediated by IL-12, without adversely affecting other aspects of the immune system. The present invention meets this need by providing methods and compounds effective at inhibiting the differentiation of T helper cells, including na\dot{i}ve T helper cells, into Th1 cells.

### SUMMARY OF THE INVENTION

The present invention provides methods for inhibiting the differentiation of T helper cells into Th1 cells. In one embodiment, the invention provides a method for inhibiting the differentiation of a T helper cell into a Th1 cell, the method comprising contacting the T helper cell with an effective amount of a compound that inhibits the activity of a hypoxia-inducible factor (HIF) prolyl hydroxylase enzyme, thereby inhibiting the differentiation of the T helper cell into the Th1 cell. In another embodiment, the invention provides a method for inhibiting the differentiation of a T helper cell into a Th1 cell in a subject, the method comprising administering to the subject an effective amount of a compound that inhibits the activity of a HIF prolyl hydroxylase enzyme, thereby inhibiting the differentiation of the T helper cell into a Th1 cell in the subject. In certain aspects, the T helper cell is a na\dot{i}ve T helper cell. In some aspects, the T helper cell is a T cell receptor (TCR)-stimulated T helper cell. In yet other aspects, the differentiation of a T helper cell into a Th1 cell is induced by IL-12, and the methods of the present invention specifically provide for inhibiting IL-12-induced differentiation of a T helper cell into a Th1 cell.

The present invention also provides for the use of a compound that inhibits the activity of a HIF prolyl hydroxylase enzyme in the manufacture of a medicament for inhibiting the differentiation of a T helper cell into a Th1 cell in a subject.

Methods for inhibiting EL-12 signaling in a T helper cell are also provided by the present invention. In one embodiment, the invention provides a method for inhibiting EL-12 signaling in a T helper cell, the method comprising contacting the T helper cell with an effective amount of a compound that inhibits the activity of a HIF prolyl hydroxylase enzyme, thereby inhibiting IL-12 signaling in the T helper cell. In other embodiments, the present invention provides methods for inhibiting IL-12-mediated T helper cell expression of IL12Rβ2, IL18R1, or IL18RAP, the method comprising contacting a T helper cell with an effective amount of a compound that inhibits the activity of a HIF prolyl hydroxylase enzyme, thereby inhibiting IL-12-mediated gene expression of IL12Rβ2, IL18R1, or IL18RAP in the T helper cell. In certain aspects, the T helper cell is a na\dot{i}ve T helper cell. In other aspects, the T helper cell is a T cell receptor (TCR)-stimulated T helper cell. Methods for inhibiting IL-12 signaling in a subject, the methods comprising administering to the subject an effective amount of a compound that inhibits HIF prolyl hydroxylase activity, are also provided herein.

Methods for inhibiting T helper cell secretion of pro-inflammatory cytokines are also provided by the present invention. In one embodiment, the present invention provides a method for inhibiting IFN-γ secretion from a T helper cell, the method comprising contacting the T helper cell with an effective amount of a compound that inhibits the activity of a HIF prolyl hydroxylase enzyme, thereby inhibiting the IFN-γ secretion from the T helper cell. In one embodiment, the present invention provides a method for inhibiting TNF-α secretion from a T helper cell, the method comprising contacting the T helper cell with an effective amount of a compound that inhibits the activity of a HIF prolyl hydroxylase enzyme, thereby inhibiting the TNF-α secretion from the T helper cell. In certain aspects, the T helper cell is a na\dot{i}ve T helper cell. In other aspects, the T helper cell is a T cell receptor (TCR)-stimulated T helper cell. In yet other aspects, the secretion of IFN-γ or TNF-α by a T helper cell is induced by IL-12, and the methods of the present invention specifically provide for inhibiting IL-12-induced IFN-γ and TNF-α secretion from a T helper cell. The invention further provides methods for inhibiting T helper cell secretion of IFN-γ and TNF-α in a subject, the methods comprising administering to the subject an effective amount of a compound that inhibits HIF prolyl hydroxylase activity.

In various embodiments, a compound used in the present methods is a structural mimetic of 2-oxoglutarate, wherein the compound inhibits the target HIF prolyl hydroxylase enzyme competitively with respect to 2-oxoglutarate and noncompetitively with respect to iron. In some embodiments, compounds of the present invention include heterocyclic carboxamides, phenanthrolines, and hydroxamates. In other embodiments, a heterocyclic carboxamide of the present invention is a pyridine carboxamide, a quinoline carboxamide, a quinolone carboxamide, an isoquinoline carboxamide, a cinnoline carboxamide, or a beta-carboline carboxamide. In other embodiments, compounds of the present invention include variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, and 4-hydroxy-pyrrolopyridine-5-carbonyl-glycines. In particular embodiments, the compound is [(4-Hydroxy-1-methyl-7-phenoxy-isoquinoline-3-carbonyl)-amino]-acetic acid (Compound A), [(4-Hydroxy-7-phenoxy-isoquinoline-3-carbonyl)-amino]-acetic acid (Compound B), [(1-Benzoyl-4-hydroxy-isoquinoline-3-carbonyl)-amino]-acetic acid (Compound C), [(8-Chloro-4-hydroxy-5-methyl-isoquinoline-3-carbonyl)-amino]-acetic acid (Compound D), {[4-Hydroxy-8-(4-methoxy-phenoxy)-1-methyl-isoquinoline-3-carbonyl]-amino}-acetic acid (Compound E), (S)-2-[(1-Cyano-4-hydroxy-8-phenoxy-isoquinoline-3-carbonyl)-amino]-propionic acid (Compound F), (S)-2-[(1-Cyano-4-hydroxy-7-phenoxy-isoquinoline-3-carbonyl)-amino]-propionic acid (Compound G), {2-[3-Bromo-2-(4-chloro-phenyl)-4-hydroxy-1-phenyl-1H-pyrrolo[2,3-c]pyridine-5-carbonyl)-amino]-acetic acid (Compound H), [(7-Cyano-4-hydroxy-1-methoxymethyl-1H-pyrrolo[2,3-c]pyridine-5-carbonyl)-amino]-acetic acid (Compound I), [(1-Benzyl-4-hydroxy-2-oxo-6-phenyl-1,2-dihydro-pyridine-3-carbonyl)-amino]-acetic acid (Compound J), [(1-Hexyl-4-hydroxy-2-oxo-6-phenyl-1,2-dihydro-pyridine-3-carbonyl)-amino]-acetic acid (Compound K), [(1-Benzyl-4-hydroxy-2-oxo-5-phenyl-1,2-dihydro-pyridine-3-carbonyl)-amino]-acetic acid (Compound L), {[4-Hydroxy-2-oxo-1-(2-trifluoromethyl-benzyl)-1,2-dihydro-pyrrolo[1,2-b]pyridazine-3-carbonyl]-amino}-acetic acid (Compound M), [(1-Hexyl-4-hydroxy-2-oxo-1,2-dihydro-pyrrolo[1,2-b]pyridazine-3-carbonyl)-amino]-acetic acid (Compound N), [(7-Chloro-3-hydroxy-quinoline-2-carbonyl)-amino]-acetic acid (Compound O), [(3-Benzyloxy-7-chloro-quinoline-2-carbonyl)-amino]-acetic acid (Compound P), [(3-Hydroxy-6-phenoxy-quinoline-2-carbonyl)-amino]-acetic acid (Compound Q), [(2-tert-Butyl-7-hydroxy-4-phenethyl-thiazolo[4,5-c]pyridine-6-carbonyl)-amino]-acetic acid (Compound R), (S)-2-[(7-Hydroxy-4-methyl-2-phenyl-thiazolo[4,5-c]pyridine-6-carbonyl)-amino]-propionic acid (Compound S), [(1-Chloro-4-hydroxy-6-phenylsulfanyl-isoquinoline-3-carbonyl)-amino]-acetic acid (Compound T), [(1-Benzyl-2,3-dichloro-7-hydroxy-4-methyl-1H-pyrrolo[3,2-c]pyridine-6-carbonyl)-amino]-acetic acid (Compound U), or [(1-Benzyl-5,6,7-trichloro-4-hydroxy-2-oxo-1,2-dihydro-pyrrolo[1,2-b]pyridazine-3-carbonyl)-amino]-acetic acid (Compound V).

Compounds for use in the present invention are compounds that inhibit HIF prolyl hydroxylase activity. A compound that inhibits HIF prolyl hydroxylase activity is any compound that reduces or otherwise inhibits the activity of at least one HIF prolyl hydroxylase enzyme. Various compounds that inhibit HIF prolyl hydroxylase have been identified and are suitable for use in the methods and medicaments as claimed in the present invention.

In one embodiment, a compound for use in the present methods and medicaments is a pyridine-2-carboxamide, a pyridazine-3-carboxamide, a quinoline-2-carboxamide, an isoquinoline-3-carboxamide or ester thereof as described in European Patent Nos. EP0650960 and EP0650961. In another embodiment, a compound for use in the present methods and medicaments is a pyridine-2-carboxamide as described in U.S. Patent Application Publication No. 2007/0299086. In yet another embodiment, a compound for use in the present methods and medicaments is a pyridine-2-carboxamidoester, a pyridazine-3-carboxamidoester, or an isoquinoline-3-carboxamidoester as described in U.S. Pat. No. 5,658,933.

In some embodiments, a compound for use in the present methods and medicaments is a pyridine-2-carboxamide, a pyridizine-3-carboxamide, or a quinoline-2-carboxamide as described in U.S. Pat. No. 5,620,995. In another embodiment, a compound for use in the methods and medicaments of the present invention is a 3-hydroxypyridine-2-carboxamidoester as described in U.S. Pat. No. 6,020,350; a sulfonamidocarbonylpyridine-2-carboxamide as described in U.S. Pat. No. 5,607,954; or a sulfonamidocarbonyl-pyridine-2-carboxamide or a sulfonamidocarbonyl-pyridine-2-carboxamide ester as described in U.S. Pat. Nos. 5,610,172 and 5,620,996. In yet another embodiment, a compound for use in the present methods and medicaments is a quinoline-2-carboxamide as described in U.S. Pat. Nos. 5,719,164 and 5,726,305.

In other embodiments, a compound for use in the present methods and medicaments is an isoquinoline-3-carboxamide as described in U.S. Pat. Nos. 6,093,730 and 7,323,475. In another embodiment, a compound for use in the present methods and medicaments is an isoquinoline-3-carboxamide as described in U.S. Patent Application Publication No. 2007/0298104. In still another embodiment, a compound for use in the present methods and medicaments is a beta-carboline-3-carboxamide, a pyrrolo[3,2-c]pyridine-6-carboxamide, a pyrrolo[2,3-c]pyridine-5-carboxamide, a thiazolo[4,5-c]pyridine-6-carboxamide, or a thiazolo[5,4-c]pyridine-6-carboxamide as described in U.S. Patent Application Publication No. 2008/0004309.

In one embodiment, a compound for use in the present methods and medicaments is a thieno[3,2-c]pyridine-6-carboxamide or a thieno[2,3-c]pyridine-5-carboxamide as described in U.S. Patent Application Publication No. 2006/0199836. In another embodiment, a compound for use in the present methods and medicaments is a 2,4-dioxo-1,2,3,4-tetrahydro-pyrimidine-5-carboxamide or a 4-oxo-2-thioxo-1,2,3,4-tetrahydro-pyrimidine-5-carboxamide as described in International Publication No. WO 2007/150011. In yet another embodiment, a compound for use in the present methods and medicaments is a 6-oxo-1,6-dihydro-pyrimidine-5-carboxamide as described in U.S. Patent Application Publication No. 2008/0171756.

In some embodiments, a compound for use in the present methods and medicaments is a 2-oxo-1,2-dihydro-quinoline-3-carboxamide as described in International Publication No. WO 2007/038571 and U.S. Patent Application Publication No. 2007/0249605. In other embodiments, a compound for use in the present methods and medicaments is a 2-oxo-1,2-dihydro-[1,8]naphthyridine-3-carboxamide, a 2-oxo-1,2-dihydro-[1,6]naphthyridine-3-carboxamide, or a 6-oxo-5,6-dihydro-pyrido[2,3-b]pyrazine-7-carboxamide as described in International Publication Nos. WO 2007/103905, WO 2008/076425, and WO 2008/130527. In yet another embodiment, a compound for use in the present methods and medicaments is a 6-oxo-6,7-dihydro-thieno[2,3-b]pyridine-5-carboxamide, a 5-oxo4,5-dihydro-thieno[3,2-b]pyridine-6-carboxamide, or a 6-oxo-6,7-dihydro-pyrazolo[3,4-b]pyridine-5-carboxamide as described in International Publication No. WO 2007/136990.

In one embodiment, a compound for use in the present methods and medicaments is a 3-oxo-2,3-dihydro-pyridazine-4-carboxamide as described in U.S. Patent Application Publication No. 2008/0214549. In other embodiments, a compound for use in the present methods and medicaments is a 3-oxo-3,4-dihydro-naphthalene-2-carboxamide, a 7-oxo-7,8-dihydro-quinoline-6-carboxamide, or a 7-oxo-7,8-dihydro-isoquinoline-6-carboxamide as described in International Publication No. WO 2008/076427. In another embodiment, a compound for use in the present methods and medicaments is a 3-hydroxy-1-oxo-1H-indene-2-carboxamide as described in International Publication No. WO 2008/130508.

In another embodiment, a compound for use in the present methods and medicaments is a 4-oxo-[1,10]-phenanthroline as described in U.S. Pat. Nos. 5,916,898 and 6,200,974, and International Publication No. WO 99/21860. In one aspect, a 4-oxo-[1,10]-phenanthroline is 4-oxo-1,4-dihydro-[1,10]phenanthroline-3-carboxylic acid (see, e.g., Seki et al. (1974) Chem Abstracts 81:424, No. 21).

In one embodiment, a compound for use in the present methods and medicaments is a hydrozone as described in U.S. Pat. No. 6,660,737. In other embodiments, a compound for sue in the present methods and medicaments is a dihydropyrazole or a dihydropyrozolone as described in U.S. Pat. No. 6,878,729 and International Publication No. WO 2008/049539. In another embodiment, a compound for use in the present methods and medicaments is a dipyridyl dihyropyrazones as described in International Publication No. WO 2006/114213. In other embodiments, a compound for use in the present methods and medicaments is a spiroindalone as described in International Publication No. WO 2008/144266.

In various embodiments, compounds for use in the present invention are selected from the group consisting of 2-oxoglutarate mimetics, iron chelators, and proline analogs. In preferred embodiments, the compound used in the methods and medicaments of the present invention is a 2-oxoglutarate structural mimetic. In particular embodiments, the compound used in the methods and medicaments of the present invention is a 2-oxoglutarate structural mimetic that inhibits HIF prolyl hydroxylase competitively with respect to 2-oxoglutarate and noncompetitively with respect to iron.

A compound for use in the methods and medicaments of the present invention is, in various embodiments, a cyclic carboxamide. In one aspect of the present embodiment, the cyclic carboxamide is a carbonyl glycine. In other aspects of the present embodiment, the carboxamide is replaced by a carbonyl proprionic acid. In some embodiments of the present invention, the compound used in the methods and medicaments of the present invention is a carbocyclic carboxamide.

In one embodiment, cyclic carboxamides suitable for use in the present invention are heterocyclic carboxamides. In certain embodiments, a compound of the present invention is a heterocyclic carboxamide having a heterocyclic group selected from the group consisting of: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In preferred embodiments, the heterocyclic group is a single ring selected from the group consisting of a pyridine, a pyridinone, a pyradizine, a pyridazinone, a pyrimidine, and a pyrimidinone ring. In other preferred embodiments, the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

A particularly preferred compound of the present invention is a heterocyclic carbonyl glycine. In successive embodiments, the heterocyclic carbonyl glycine suitable for use in the present invention is a heterocyclic carbonyl glycine having a heterocyclic group that is selected from the following list: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In certain preferred embodiments, the heterocyclic carbonyl glycine suitable for use in the present invention is a heterocyclic carbonyl glycine having a heterocyclic group, wherein the heterocyclic group is a single ring selected from the following list: a pyridine, a pyridinone, a pyradizine, a pyridazinone, a pyrimidine, and a pyrimidinone ring. In other preferred embodiments, the heterocyclic carbonyl glycine suitable for use in the present invention is a heterocyclic carbonyl glycine having a heterocyclic group, wherein the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

In one embodiment, a compound for use in the present methods and medicaments is an isoquinoline carbonyl glycine; preferably, an isoquinoline-3-carbonyl-glycine or a 4-hydroxy-isoquinoline-3-carbonyl glycine. In particular embodiments, a compound for use in the present methods and medicaments is {[4-Hydroxy-7-(4-methoxy-phenoxy)-isoquinoline-3-carbonyl]-amino}-acetic acid (Compound A); [(4-Hydroxy-1-methyl-7-phenoxy-isoquinoline-3-carbonyl)-amino]-acetic acid (Compound B); or {[1-Cyano-6-(2,6-dimethyl-phenoxy)-4-hydroxy-isoquinoline-3-carbonyl]-amino}-acetic acid (Compound C).

In other embodiments, a compound for use in the present methods and medicaments is a HIF prolyl hydroxylase inhibitor compound of Formula I:

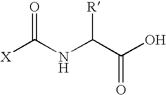

wherein X is an optionally substituted cyclic moiety and R′ is hydrogen or (C₁-C₄)-alkyl. In particular embodiments, the cyclic moiety is a hetercyclic moiety and R′ is hydrogen. Such HIF prolyl hydroyxlase inhibitors include, but are not limited to, variously substituted pyridine-2-carbonyl-glycines, pyridazine-3-carbonyl-glycines, quinoline-2-carbonyl-glycines, 2-oxo-1,2-dihydro-quinoline-3-carbonyl-glycines, 2-oxo-1,2-dihydro-naphthyridine-3-carbonyl-glycines, 6-oxo-4,6-dihydro-pyridopyrazine-7-carbonyl-glycines, isoquinoline-3-carbonyl-glycines, cinnoline-3-carbonyl-glycines, thienopyridine-6-carbonyl-glycines, thienopyridine-5-carbonyl-glycines, thiazolopyridine-6-carbonyl-glycines, thiazolopyridine-5-carbonyl-glycines, hydroxy-pyrrolopyridine-6-carbonyl-glycines, and pyrrolopyridine-5-carbonyl-glycines.

In another embodiment, a compound for use in the methods and medicaments of the present invention is a compound of Formula II:

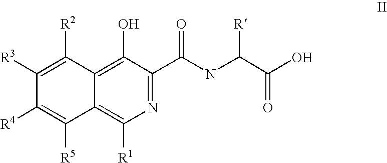

wherein:
R′ is selected from hydrogen and (C₁-C₄)-alkyl;
R¹, R², R³, R⁴ and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido, (C₆-C₁₂)-heteroaryl, (C₇-C₁₆)-heteroaralkyl; where an aryl or heteroaryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂Cl, and —OCF₂—CHFCl;
x is 0 to 3;
f is 1 to 8; and
is 0 or 1 to (2f+1);
or a pharmaceutically acceptable salt, single stereoisomer, mixture of stereoisomers, ester, or prodrug thereof.

In some embodiments, a compound for use in the present invention is a compound of Formula II wherein:
R′ is hydrogen or (C₁-C₃)-alkyl;
R¹ is selected from hydrogen, halo, (C₁-C₃)-alkyl, cyano, or arylacyl;
R² is hydrogen or (C₁-C₃)-alkyl;
R³ is hydrogen or arylsulfanyl;
R⁴ is hydrogen or aryloxy; and
R⁵ is selected from hydrogen, halo, aryloxy, or aryloxy substituted with (C₁-C₃)-alkoxy.

In other embodiments, a compound for use in the present invention is a compound of Formula II wherein:
R′ is hydrogen or methyl;
R¹ is selected from hydrogen, chloro, methyl, cyano, or benzoyl;
R² is selected from hydrogen or methyl;
R³ is hydrogen or phenylsulfanyl;
R⁴ is selected from hydrogen or phenoxy; and
R⁵ is selected from hydrogen, chloro, phenoxy, or 4-methoxy-phenoxy.

In various embodiments, a compound for use in the methods and medicaments of the present invention is a compound of Formula III:

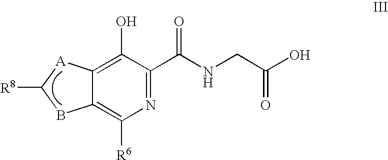

wherein:
one of A or B is ═C(R⁷)— and the other is —N(R⁹)—;
[symbol] independently represents a single or a double bond;
R⁶ is selected from the group consisting of hydrogen, halo, cyano, (C₁-C₃)-alkyl, and aryl;
R⁷ is selected from the group consisting of hydrogen, halo, cyano, (C₁-C₆)-alkyl, and aryl, wherein the aryl is optionally substituted by one or two halo;
   R⁸ is selected from the group consisting of hydrogen, halo, cyano, (C₁-C₆)-alkyl, trifluoromethyl, and aryl optionally substituted with halo; and
R⁹ is selected from the group consisting of hydrogen, (C₁-C₁₀)-alkyl, (C₁-C₃)-alkyl-(C₁-C₁₀)-alkyl, (C₁-C₃)-alkoxy-(C₁-C₆)-alkyl, (C₄-C₆)-cycloalkyl, (C₄-C₆)-cycloalkyl-(C₁-C₃)-alkyl, aryl, (C₇-C₁₂)-aralkyl, aryl-aralkyl, and heteroaralkyl; where in each case an aryl or heteroaryl may be optionally substituted by one or two halo, trifluoromethyl, or (C₁-C₄)-alkoxy;
or pharmaceutically acceptable salts, single stereoisomers, mixtures of stereoisomers, esters, or prodrugs thereof.

In some embodiments, a compound for use in the present invention is a compound of Formula III wherein:
A is ═C(R⁷)—;
B is —N(R⁹)—;
R⁶ is selected from hydrogen or cyano;
R⁷ is selected from hydrogen or halo;
R⁸ is selected from hydrogen or aryl optionally substituted with halo; and
R⁹ is selected from (C₁-C₃)-alkoxy-(C₁-C₃)-alkyl.

In further embodiments, a compound for use in the present invention is a compound of Formula Ell wherein:
A is ═C(R⁷)—;
B is —N(R⁹)—;
R⁶ is selected from hydrogen or cyano;
R⁷ is selected from hydrogen or bromo;
R⁸ is selected from hydrogen or 4-chloro-phenyl; and
R⁹ is selected from methoxymethyl or phenyl.

In yet embodiments of the present invention, a compound suitable for use in the claimed methods and medicaments is a compound of Formula III wherein:
A is —N(R⁹)—;
B is ═C(R⁷)—;
R⁶ is (C₁-C₃)-alkyl;
R⁷ is halogen;
R⁸ is halogen; and
R⁹ is aralkoxy.

In other embodiments, a compound for use in the methods and medicaments of the present invention is a compound of Formula III wherein:
R⁶ is methyl;
R⁷ is chloro;
R⁸ is chloro; and
R⁹ is benzyl.

It is contemplated in some embodiments of the present invention that a compound for use in the methods and medicaments of the invention is a compound represented by Formula IV,

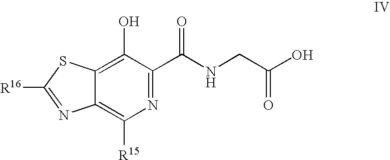

wherein:
R¹⁵ is selected from the group consisting of hydrogen, cyano, acetyl, (C₁-C₆)-alkyl, (C₁-C₄)-alkynyl, (C₇-C₁₂)-aralkyl, (C₇-C₁₂)-aralkenyl, (C_{1 -C}₆)-alkyl-sulfanyl-(C₁-C₃)-alkyl, aryl, heterocyclyl, and heteroaryl; wherein each substituent is optionally substituted with halo or cyano; and
R¹⁶ is selected from the group consisting of (C₁-C₆)-alkyl, (C₆-C₁₂)-aryl optionally substituted with one or two substituents each independently selected from cyano, (C₁-C₄)-alkyl, trifluoromethyl, halo, (C₁-C₄)-alkoxy, aryl, or aryloxy; aryloxy, heteocyclyl, and heteroaryl optionally substituted with halo, (C₁-C₄)-alkoxy, aryloxy, or arylsulfanyl;
or pharmaceutically acceptable salts, single stereoisomers, mixtures of stereoisomers, esters, or prodrugs thereof.

In certain embodiments, a compound for use in the methods and medicaments of the present invention is a compound of Formula IV wherein:
R¹⁵ is (C₁-C₃)-alkyl or (C₇-C₁₂)-aralkyl; and
R¹⁶ is selected from (C₁-C₆)-alkyl or (C₆-C₁₂)-aryl.

In other embodiments, a compound for use in the methods and medicaments of the present invention is a compound of Formula IV, wherein:
R¹⁵ is selected from methyl or phenethyl; and
R¹⁶ is selected from t-butyl or phenyl.

It is also contemplated that in certain embodiments a compound suitable for use in the present methods and medicaments is a compound of Formula V:

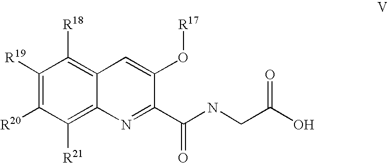

wherein:
R¹⁷ is is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
R¹⁸, R¹⁹, R²⁰, and R²¹ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-c₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C ₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido, (C₆-C₁₂)-heteroaryl, (C₇-C₁₆)-heteroaralkyl; where an aryl or heteroaryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂CI, and —OCF₂—CHFCl;
x is 0 to 3;
f is 1 to 8; and
g is 0 or 1 to (2f+1);
or a pharmaceutically acceptable salt, single stereoisomer, mixture of stereoisomers, ester, or prodrug thereof.

In further embodiments, a compound for use in the present invention is a compound of Formula V wherein:
R¹⁷ is selected from hydrogen or aralkoxy;
R¹⁸ and R²¹ are hydrogen;
R¹⁹ is selected from hydrogen or aryloxy; and
R²⁰ is selected from hydrogen or halo.

In other embodiments, a compound for use in the present invention is a compound of Formula V wherein:
R¹⁷ is selected from hydrogen or benzyloxy;
R¹⁸ and R²¹ are hydrogen;
R¹⁹ is selected from hydrogen or phenoxy; and
R²⁰ is selected from hydrogen or chloro.

It is contemplated that a compound for use in the methods and medicaments of the present invention is in various embodiments a compound of Formula VI:

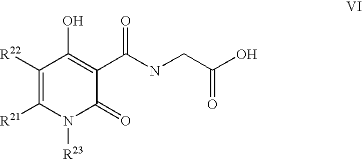

wherein:
R²¹ is selected from hydrogen or aryl;
R²² is selected from hydrogen or aryl; and
R²³ is selected from (C₁-C₁₀)-alkyl or aralkyl.

In some embodiments, a compound for use in the present invention is a compound of Formula VI wherein:
R²¹ is selected from hydrogen or phenyl;
R²² is selected from hydrogen or phenyl; and
R²³ is selected from hexyl or benzyl.

In other embodiments, a compound for use in the present methods and medicaments is a compound of Formula VII:

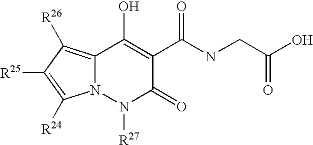

wherein:
R²⁴, R²⁵, and R²⁶ are each independently selected from hydrogen or halogen; and
R²⁷ is (C₇-C₁₂)-aralkyl optionally substituted on the aryl with one or two substituents selected from the group consisting of halo, trifluoromethyl, and (C₁-C₃)-alkoxy.

In certain embodiments, a compound for use in the present invention is a compound of Formula VII wherein:
R²⁴ is selected from hydrogen or halo;
R²⁵ is selected from hydrogen or halo;
R²⁶ is selected from hydrogen or halo; and
R²⁷ is selected from (C₁-C₁₀)-alkyl or (C₇-C₁₂)-aralkyl, wherein the aryl is optionally substituted with trifluoromethyl.

In further embodiments, a compound for use in the present invention is a compound of Formula VII wherein:
R²⁴ is selected from hydrogen or chloro;
R²⁵ is selected from hydrogen or chloro;
R²⁶ is selected from hydrogen or chloro; and
R²⁷ is selected from hexyl, benzyl, or 2-trifluoromethyl-benzyl.

Pharmaceutical compositions or medicaments effective for use in any of the present methods are provided herein. In various embodiments, the compositions comprise an effective amount of a compound that inhibits the activity of a HIF prolyl hydroxylase and an acceptable carrier.

These and other embodiments of the present invention will readily occur to those of skill in the art in light of the disclosure herein, and all such embodiments are specifically contemplated.

### BRIEF DESCRIPTION OF THE DRAWINGS

FIGS. 1A and 1B set forth data showing a compound of the present invention decreased IFN-γ and TNF-α secretion from T cells.

FIGS. 2A and 2B set forth data showing a compound of the present invention did not reduce T cell number or viability.

FIGS. 3A, 3B, and 3C set forth data showing a compound of the present invention decreased IL-12-mediated expression of IL12Rb2, IL18R1, and IL18RAP in T cells.

FIGS. 4A and 4B set forth data showing a compound of the present invention decreased IL-12-mediated IFN-γ secretion, but not IL-4-mediated IL-5 secretion, from T cells.

FIGS. 5A and 5B set forth data showing a compound of the present invention inhibited Th1 cell differentiation and IL-12-mediated IFN-γ secretion from T cells.

FIGS. 6A and 6B set forth data showing a compound of the present invention inhibited IL-12-mediated IFN-γ and TNF-α secretion ex vivo from T cells activated in vivo.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a HIF prolyl hydroxylase enzyme” may include a plurality of such enzymes; a reference to a “compound that inhibits the activity of a hypoxia-inducible factor prolyl hydroxylase enzyme” may be a reference to one or more compounds that inhibits the activity of a hypoxia-inducible factor prolyl hydroxylase enzyme; and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Hardman, J. G., Limbird, L. E., and Gilman, A. G., eds. (2001) The Pharmacological Basis of Therapeutics, 10^{th} ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2^{nd} ed., Springer Verlag.

The section headings are used herein for organizational purposes only, and are not to be construed as in any way limiting the subject matter described herein.

### Invention

The present invention relates to the discovery that inhibiting hypoxia-inducible factor (HIF) hydroxylase is effective at inhibiting the differentiation of T helper cells into Th1 cells. In one embodiment, the present invention provides a method for inhibiting the differentiation of T helper cells into Th1 cells, the method comprising contacting a T helper cell with an effective amount of a compound that inhibits the activity of a HIF prolyl hydroxylase enzyme, thereby inhibiting the differentiation of the T helper cell into a Th1 cell. In certain embodiments, the T helper cell in these methods is a na\dot{i}ve T helper cell.

In another embodiment, the present invention provides a method for inhibiting the differentiation of a T helper cell into a Th1 cell in a subject, the method comprising administering to the subject an effective amount of a compound that inhibits the activity of a HIF prolyl hydroxylase enzyme, thereby inhibiting the differentiation of the T helper cell into a Th1 cell in the subject. The invention also provides compounds for use in manufacturing a medicament for inhibiting the differentiation of a T helper cell into a Th1 cell in a subject, wherein the compound inhibits the activity of a HIF prolyl hydroxylase enzyme. In certain embodiments, the T helper cell in these methods is a na\dot{i}ve T helper cell.

Th1 cells and Th2 cells are subsets of CD4+T cells derived from a common CD4+ na\dot{i}ve T helper cell (i.e., a Th0 cell). During the initial encounter with antigen, the differentiation of na\dot{i}ve T helper cells into Th1 cells and Th2 cells is controlled primarily by the opposing actions of two key cytokines, IL-12 and IL-4, which induce the differentiation of na\dot{i}ve T helper cells into Th1 cells and Th2 cells, respectively. In response to IL-12, Th1 cells secrete various cytokines associated with inflammation, such as, for example, IFN-γ, TNF-α, TNF-β, and IL-2. IFN-γ is an important component to the inflammatory response, contributing to phagocytic cell activation, up-regulation of MHC expression of antigen presenting cells, and generally associated with inflammatory and immune responses. (See, e.g., Heremann et al. (1989) Lymphokine Research 8:329-333.)

The present invention shows that compounds effective at inhibiting HIF prolyl hydroxylase enzyme activity inhibit IL-12-induced T helper cell production of IFN-γ. Therefore, in one embodiment, the present invention provides a method for inhibiting IL-12-mediated induction of IFN-γ synthesis in a subject, the method comprising administering to the subject an effective amount of a compound that inhibits the activity of a HIF prolyl hydroxylase enzyme, thereby inhibiting IL-12-mediated induction of IFN-γ synthesis in the subject. In another embodiment, the present invention provides a method for inhibiting IL-12-mediated induction of IFN-γ synthesis in a T helper cell, the method comprising contacting a T helper cell with an effective amount of a compound that inhibits the activity of a HIF prolyl hydroxylase enzyme, thereby inhibiting IL-12-mediated induction of IFN-γ synthesis in the T helper cell. Methods for inhibiting the induction of IFN-γ synthesis in a T helper cell, the method comprising contacting the T helper cell, in an amount sufficient to inhibit IL-12-induced production of IFN-γ, with a compound that inhibits the activity of a HIF prolyl hydroxylase enzyme, are specifically contemplated. In certain aspects, the T helper cell is a Th1 cell. In other aspects, the T helper cell is a na\dot{i}ve T helper cell.

The present invention also shows that compounds effective at inhibiting HIF prolyl hydroxylase enzyme activity inhibit IL-12-induced T helper cell production of TNF-α. Therefore, in one embodiment, the present invention provides a method for inhibiting IL-12-mediated induction of TNF-α synthesis in a subject, the method comprising administering to the subject an effective amount of a compound that inhibits the activity of a HIF prolyl hydroxylase enzyme, thereby inhibiting IL-12-mediated induction of TNF-α synthesis in the subject. In another embodiment, the present invention provides a method for inhibiting IL-12-mediated induction of TNF-α synthesis in a T helper cell, the method comprising contacting a T helper cell with an effective amount of a compound that inhibits the activity of a HIF prolyl hydroxylase enzyme, thereby inhibiting IL-12-mediated induction of TNF-α synthesis in the T helper cell. Methods for inhibiting the induction of TNF-α synthesis in a T helper cell, the method comprising contacting the T helper cell, in an amount sufficient to inhibit IL-12-induced production of TNF-α, with a compound that inhibits the activity of a HEF prolyl hydroxylase enzyme, are specifically contemplated. In certain aspects, the T helper cell is a Th1 cell. In other aspects, the T helper cell is a na\dot{i}ve T helper cell.

The present invention also provides methods and compounds useful for inhibiting IL-12 signaling in T helper cells. In some embodiments, the present invention provides a method for inhibiting IL-12 signaling in a T helper cell, the method comprising contacting a T helper cell with an effective amount of a compound that inhibits the activity of a HIF prolyl hydroxylase enzyme, thereby inhibiting IL-12 signaling in the T helper cell. In certain aspects, the T helper cell is a Thll cell. In other aspects, the T helper cell is a na\dot{i}ve T helper cell.

By inhibiting IL-12 signaling in T helper cells, the present methods and compounds are useful for inhibiting IL-12-mediated gene expression in these cells. In particular, the present invention provides a method for inhibiting IL-12-mediated gene expression in a T helper cell, the method comprising contacting the T helper cell with an effective amount of a compound that inhibits the activity of a HIF prolyl hydroxylase enzyme, thereby inhibiting IL-12-mediated gene expression in the T helper cell.

The present invention shows that compounds effective at inhibiting HIF prolyl hydroxylase enzyme activity inhibit the expression of three IL-12-induced genes: interleukin-18 receptor beta 2 (IL-18Rβ2); interleukin-18 receptor 1 (IL-18R1), also known as interleukin-18 receptor alpha chain (IL-18RA, IL-18Ra, IL-18Rα) and interleukin-1 receptor-related protein (IL-1RRP); and interleukin-18 receptor-associated protein (IL-18RAP), also known as interleukin-18 receptor beta chain (IL-18RB, IL-18Rβ) and accessory protein-like (ACPL). (See, e.g., Saremeva et al. (2000) J Immunol 165:1933-1938; Rogge et al. (1997) J Exp Med 185:825-831; and Nakahira et al. (2001) J Immunol 167:1306-1312.) Therefore, in one embodiment, the methods and compounds of the present invention are useful for inhibiting T helper cell expression of IL12Rβ2, IL18R1, and IL18RAP, the method comprising contacting a T helper cell with an effective amount of a compound that inhibits the activity of a HIF prolyl hydroxylase enzyme, thereby inhibiting IL-12-mediated gene expression of IL12Rβ2, IL18R1, and IL18RAP in the T helper cell. In certain aspects, the T helper cell is a Th1 cell. In other aspects, the T helper cell is a na\dot{i}ve T helper cell.

In another embodiment, the present methods and compounds are useful for and effective at inhibiting T helper cell expression of IL12Rβ2, IL18R1, and IL18RAP in a subject, the method comprising administering to the subject an effective amount of a compound that inhibits the activity of a HIF prolyl hydroxylase enzyme, thereby inhibiting T helper cell expression of IL12Rβ2, IL18R1, and IL18RAP in the subject. In certain aspects, the inhibition of T helper cell expression of IL12Rβ2, IL18R1, and IL18RAP is inhibition of IL-12-induced gene expression of IL12Rβ2, IL18R1, and IL18RAP expression in T helper cells in the subject. In certain aspects, the T helper cell is a Th1 cell. In other aspects, the T helper cell is a na\dot{i}ve T helper cell.

### Subjects

The present invention relates to methods and compounds usefuil for inhibiting T helper cell differentiation in a subject by administering to the subject an effective amount of a compound that inhibits the activity of a hypoxia-inducible factor (HIF) prolyl hydroxylase.

The invention is applicable to a variety of different organisms, including, for example, vertebrates, large animals, and primates. In a preferred embodiment, the subject is a mammalian subject, and in a most preferred embodiment, the subject is a human subject. However, although medical applications with humans are clearly foreseen, veterinary applications are also envisaged herein.

The methods and compounds of the present invention are particularly suitable for a subject who would benefit from inhibition of T helper cell differentiation, in particular, from inhibition of T helper cell differentiation into Th1 cells, such as, for example, a subject having or at risk for having a cell-mediated immune response. In certain embodiments, a subject suitable for treatment with the present methods and compounds is a subject having or is at risk for having a cell-mediated immune response. In certain aspects, a suitable subject for the present methods is one in which the subject would benefit from a reduction in cell-mediated immune responses (i.e., Th1-mediated responses) without affecting humoral immune responses (i.e., Th2-mediated responses).

In other embodiments, a subject suitable for treatment with the present methods and compounds is a subject having or at risk for having a pathological Th1 cell response, such as, for example, a subject having or at risk for having an organ-specific or systemic autoimmune condition, including a chronic inflammatory disease or a delayed type hypersensitivity reaction.

### Compounds

Compounds for use in the methods or medicaments provided herein are inhibitors of hypoxia-inducible factor (HIF) prolyl hydroxylase enzymes. The term “HIF prolyl hydroxylase,” as used herein, refers to any enzyme that is capable of hydroxylating a proline residue within an alpha subunit of HIF. Such HIF prolyl hydroxylases include protein members of the EGL-9 (EGLN) 2-oxoglutarate- and iron-dependent dioxygenase family described by Taylor (2001) Gene 275:125-132; and characterized by Aravind and Koonin (2001) Genome Biol 2:RESEARCH0007; Epstein et al. (2001) Cell 107:43-54; and Bruick and McKnight (2001) Science 294:1337-1340. Examples of HIF prolyl hydroxylases include human SM-20 (EGLN1) (GenBank Accession No. AAG33965; Dupuy et al. (2000) Genomics 69:348-54), EGLN2 isoform 1 (GenBank Accession No. CAC42510), EGLN2 isoform 2 (GenBank Accession No. NP_{—}060025), and EGLN3 (GenBank Accession No. CAC42511; Taylor, supra); mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF prolyl hydroxylase may include Caenorhabditis elegans EGL-9 (GenBank Accession No. AAD56365) and Drosophila melanogaster CG1114 gene product (GenBank Accession No. AAF52050). The term “HIF prolyl hydroxylase” also includes any active fragment of the foregoing full-length proteins.

A compound that inhibits the activity of a HIF prolyl hydroxylase enzyme refers to any compound that reduces or otherwise modulates the activity of at least one HIF prolyl hydroxylase enzyme. A compound may additionally show inhibitory activity toward one or more other 2-oxoglutarate- and iron-dependent dioxygenase enzymes, e.g. factor inhibiting HIF (FIH; GenBank Accession No. AAL27308), procollagen prolyl 4-hydroxylase (cP4H), etc. In particular embodiments, compounds used in the present methods and medicaments provided herein are structural mimetics of 2-oxoglutarate, wherein the compound inhibits the target HIF prolyl hydroxylase enzyme competitively with respect to 2-oxoglutarate and noncompetitively with respect to iron. Examples of compounds that may be used in the methods and medicaments provided herein can be found, e.g., in Majamaa et al. (1984) Eur. J. Biochem. 138:239-245; Majamaa et al. (1985) Biochem. J. 229:127-133; Kivirikko, and Myllyharju (1998) Matrix Biol. 16:357-368; Bickel et al. (1998) Hepatology 28:404-411; Friedman et al. (2000) Proc. Natl. Acad. Sci. USA 97:4736-4741; Franklin (1991) Biochem. Soc. Trans. 19):812-815; and Franklin et al. (2001) Biochem. J. 353:333-338. Additionally, compounds that inhibit HIF prolyl hydroxylase enzyme activity or that stabilize HIFa have been described in, e.g., International Publication Nos. WO 03/049686, WO 02/074981, WO 03/080566, WO 2004/108681, WO 2006/094292, WO 2007/038571, WO 2007/090068, WO 2007/070359, WO 2007/103905, and WO 2007/115315.

Examples of additional compounds that may be used in the methods and medicaments provided herein include, but are not limited to, heterocyclic carboxamides (including pyridine carboxamides, quinoline carboxamides, isoquinoline carboxamides, a quinolone carboxamide, cinnoline carboxamides, or beta-carboline carboxamides), phenanthrolines, hydroxamates, and variously substituted 3-hydroxy-pyridine-2-carbonyl-glycines, 4-hydroxy-pyridazine-3-carbonyl-glycines, 3-hydroxy-quinoline-2-carbonyl-glycines, 4-hydroxy-2-oxo-1,2-dihydro-quinoline-3-carbonyl-glycines, 4-hydroxy-2-oxo-1,2-dihydro-naphthyridine-3-carbonyl-glycines, 8-hydroxy-6-oxo4,6-dihydro-pyridopyrazine-7-carbonyl-glycines, 4-hydroxy-isoquinoline-3-carbonyl-glycines, 4-hydroxy-cinnoline-3-carbonyl-glycines, 7-hydroxy-thienopyridine-6-carbonyl-glycines, 4-hydroxy-thienopyridine-5-carbonyl-glycines, 7-hydroxy-thiazolopyridine-6-carbonyl-glycines, 4-hydroxy-thiazolopyridine-5-carbonyl-glycines, 7-hydroxy-pyrrolopyridine-6-carbonyl-glycines, and 4-hydroxy-pyrrolopyridine-5-carbonyl-glycines. In particular embodiments, the compound is [(4-Hydroxy-1-methyl-7-phenoxy-isoquinoline-3-carbonyl)-amino]-acetic acid (Compound A), [(4-Hydroxy-7-phenoxy-isoquinoline-3-carbonyl)-amino]-acetic acid (Compound B), [(1-Benzoyl-4-hydroxy-isoquinoline-3-carbonyl)-amino]-acetic acid (Compound C), [(8-Chloro-4-hydroxy-5-methyl-isoquinoline-3-carbonyl)-amino]-acetic acid (Compound D), {[4-Hydroxy-8-(4-methoxy-phenoxy)-1-methyl-isoquinoline-3-carbonyl]-amino}-acetic acid (Compound E), (S)-2-[(1-Cyano-4-hydroxy-8-phenoxy-isoquinoline-3-carbonyl)-amino]-propionic acid (Compound F), (S)-2-[(1-Cyano-4-hydroxy-7-phenoxy-isoquinoline-3-carbonyl)-amino]-propionic acid (Compound G), {2-[3-Bromo-2-(4-chloro-phenyl)-4-hydroxy-1-phenyl-1H-pyrrolo[2,3-c]pyridine-5-carbonyl)-amino]-acid (Compound H), [(7-Cyano4-hydroxy-1-methoxymethyl-1H-pyrrolo[2,3-c]pyridine-5-carbonyl)-amino]-acetic acid (Compound I), [(1-Benzyl-4-hydroxy-2-oxo-6-phenyl-1,2-dihydro-pyridine-3-carbonyl)-amino]-acetic acid (Compound J), [(1-Hexyl-4-hydroxy-2-oxo-6-phenyl-1,2-dihydro-pyridine-3-carbonyl)-amino]-acetic acid (Compound K), [(1-Benzyl-4-hydroxy-2-oxo-5-phenyl-1,2-dihydro-pyridine-3-carbonyl)-amino]-acetic acid (Compound L), {[4-Hydroxy-2-oxo-1-(2-trifluoromethyl-benzyl)-1,2-dihydro-pyrrolo[1,2-b]pyridazine-3-carbonyl]-amino}-acetic acid (Compound M), [(1-Hexyl-4-hydroxy-2-oxo-1,2-dihydro-pyrrolo[1,2-b]pyridazine-3-carbonyl)-amino]-acetic acid (Compound N), [(7-Chloro-3-hydroxy-quinoline-2-carbonyl)-amino]-acetic acid (Compound O), [(3-Benzyloxy-7-chloro-quinoline-2-carbonyl)-amino]-acetic acid (Compound P), [(3-Hydroxy-6-phenoxy-quinoline-2-carbonyl)-amino]-acetic acid (Compound Q), [(2-tert-Butyl-7-hydroxy-4-phenethyl-thiazolo[4,5-c]pyridine-6-carbonyl)-amino]-acetic acid (Compound R), (S)-2-[(7-Hydroxy4-methyl-2-phenyl-thiazolo[4,5-c]pyridine-6-carbonyl)-amino]-propionic acid (Compound S), [(1-Chloro-4-hydroxy-6-phenylsulfanyl-isoquinoline-3-carbonyl)-amino]-acetic acid (Compound T), [(1-Benzyl-2,3-dichloro-7-hydroxy-4-methyl-1H-pyrrolo[3,2-c]pyridine-6-carbonyl)-amino]-acetic acid (Compound U), or [(1-Benzyl-5,6,7-trichloro-4-hydroxy-2-oxo-1,2-dihydro-pyrrolo[1,2-b]pyridazine-3-carbonyl)-amino]-acetic acid (Compound V).

Compounds for use in the present invention are compounds that inhibit HIF prolyl hydroxylase activity. A compound that inhibits HIF prolyl hydroxylase activity is any compound that reduces or otherwise inhibits the activity of at least one HIF prolyl hydroxylase enzyme. Various compounds that inhibit HIF prolyl hydroxylase have been identified and are suitable for use in the methods and medicaments as claimed in the present invention.

Exemplary pyridine-2-carboxamides, pyridazine-3-carboxamides, quinoline-2-carboxamides, isoquinoline-3-carboxamides and esters thereof are described in European Patent Nos. EP0650960 and EP0650961. All compounds listed in EP0650960 and EP0650961, in particular, those listed in the compound claims and the final products of the working examples, are hereby incorporated into the present application by reference herein. Additional pyridine-2-carboxamides are described in U.S. Patent Application Publication No. 2007/0299086. All compounds listed in U.S. Patent Application Publication No. 2007/0299086, in particular, those listed in the compound claims and the final products of the working examples, are hereby incorporated into the present application by reference herein. Additionally, exemplary pyridine-2-carboxamidoesters, pyridazine-3-carboxamidoesters, and isoquinoline-3-carboxamidoesters are described in U.S. Pat. No. 5,658,933. All pyridine-2-carboxamidoesters, pyridazine-3-carboxarmidoesters, and quinoline-2-carboxamidesters are listed in U.S. Pat. No. 5,658,933, in particular, those listed in the compound claims and the final products of the working examples, are hereby incorporated into the present application by reference herein.

Additional pyridine-2-carboxamides, pyridizine-3-carboxamides, and quinoline-2-carboxamides are described in U.S. Pat. No. 5,620,995. All compounds listed in U.S. Pat. No. 5,620,995, in particular, those listed in the compound claims and the final products of the working examples, are hereby incorporated into the present application by reference herein. Exemplary 3-hydroxypyridine-2-carboxamidoesters are described in U.S. Pat. No. 6,020,350; sulfonamidocarbonylpyridine-2-carboxamides are described in U.S. Pat. No. 5,607,954; and sulfonamidocarbonyl-pyridine-2-carboxamides and sulfonamidocarbonyl-pyridine-2-carboxamide esters are described in U.S. Pat. Nos. 5,610,172 and 5,620,996. All compounds listed in these patents, in particular, those compounds listed in the compound claims and the final products of the working examples, are hereby incorporated into the present application by reference herein.

Exemplary quinoline-2-carboxamides are described in U.S. Pat. Nos. 5,719,164 and 5,726,305. All compounds listed in the foregoing patents, in particular, those listed in the compound claims and the final products of the working examples, are hereby incorporated into the present application by reference herein.

Exemplary isoquinoline-3-carboxamides are described in U.S. Pat. Nos. 6,093,730 and 7,323,475. All compounds listed in U.S. Pat. Nos. 6,093,730 and 7,323,475, in particular, those listed in the compound claims and the final products of the working examples, are hereby incorporated into the present application by reference herein. Particularly exemplary embodiments of isoquinoline-3-carboxamides are described in U.S. Patent Application Publication No. 2007/0298104. All compounds listed in U.S. Patent Application Publication No. 2007/0298104, in particular, those listed in the compound claims and the final products of the working examples, are hereby incorporated into the present application by reference herein.

Exemplary beta-carboline-3-carboxamides, pyrrolo[3,2-c]pyridine-6-carboxamides, pyrrolo[2,3-c]pyridine-5-carboxamides, thiazolo[4,5-c]pyridine-6-carboxamides, and thiazolo[5,4-c]pyridine-6-carboxamides are described in U.S. Patent Application Publication No. 2008/0004309. All compounds listed in U.S. Patent Application Publication No. 2008/0004309, in particular, those listed in the compound claims and the final products of the working examples, are hereby incorporated into the present application by reference herein.

Exemplary thieno[3,2-c]pyridine-6-carboxamide and thieno[2,3-c]pyridine-5-carboxamides are described in U.S. Patent Application Publication No. 2006/0199836. All compounds listed in U.S. Patent Application Publication No. 2006/0199836, in particular, those listed in the compound claims and the final products of the working examples, are hereby incorporated into the present application by reference herein.

Exemplary 2,4-dioxo-1,2,3 ,4-tetrahydro-pyrimidine-5-carboxamides and 4-oxo-2-thioxo-1,2,3,4-tetrahydro-pyrimidine-5-carboxamides are described in International Publication No. WO 2007/150011. All compounds listed in the foregoing publication, in particular, those listed in the compound claims and the final products of the working examples, are hereby incorporated into the present application by reference herein. Exemplary 6-oxo-1,6-dihydro-pyrimidine-5-carboxamides are described in U.S. Patent Application Publication No. 2008/0171756. All compounds listed in U.S. Patent Application Publication No. 2008/0171756, in particular, those listed in the compound claims and the final products of the working examples, are hereby incorporated into the present application by reference herein.

Exemplary 2-oxo-1,2-dihydro-quinoline-3-carboxamides are described in Intemational Publication No. WO 2007/038571 and U.S. Patent Application Publication No. 2007/0249605. All compounds listed in the foregoing publications, in particular, those listed in the compound claims and the final products of the working examples, are hereby incorporated into the present application by reference herein.

Exemplary 2-oxo-1,2-dihydro-[1,8]naphthyridine-3-carboxamides, 2-oxo-1,2-dihydro-[1,6]naphthyridine-3-carboxamides, and 6-oxo-5,6-dihydro-pyrido[2,3-b]pyrazine-7-carboxamides are described in International Publication Nos. WO 2007/103905, WO 2008/076425, and WO 2008/130527.

All compounds listed in the foregoing publications, in particular, those listed in the compound claims and the final products of the working examples, are hereby incorporated into the present application by reference herein.

Exemplary 6-oxo-6,7-dihydro-thieno[2,3-b]pyridine-5-carboxamides, 5-oxo-4,5-dihydro-thieno[3,2-b]pyridine-6-carboxamides, 6-oxo-6,7-dihydro-pyrazolo[3,4-b]pyridine-5-carboxamides are described in International Publication No. WO 2007/136990. All compounds listed in the foregoing publications, in particular, those listed in the compound claims and the final products of the working examples, are hereby incorporated into the present application by reference herein.

Exemplary 3-oxo-2,3-dihydro-pyridazine-4-carboxamides are described in U.S. Patent Application Publication No. 2008/0214549. All compounds listed in U.S. Patent Application Publication No. 2008/0214549, in particular, those listed in the compound claims and the final products of the working examples, are hereby incorporated into the present application by reference herein.

Exemplary 3-oxo-3,4-dihydro-naphthalene-2-carboxamides, 7-oxo-7,8-dihydro-quinoline-6-carboxamides, and 7-oxo-7,8-dihydro-isoquinoline-6-carboxamides are described in International Publication No. WO 2008/076427. All compounds listed in the foregoing publication, in particular, those listed in the compound claims and the final products of the working examples, are hereby incorporated into the present application by reference herein.

Exemplary 3-hydroxy-1-oxo-lH-indene-2-carboxamides are described in International Publication No. WO 2008/130508. All compounds listed in International Publication No. WO 2008/130508, in particular, those listed in the compound claims and the final products of the working examples, are hereby incorporated into the present application by reference herein.

Exemplary 4-oxo-[1,10]-phenanthrolines are described in U.S. Pat. Nos. 5,916,898 and 6,200,974, and International Publication No. WO 99/21860. All compounds listed in the foregoing patents and publication, in particular, those listed in the compound claims and the final products of the working examples, are hereby incorporated into the present application by reference herein. An exemplary 4-oxo-[1,10]-phenanthroline is 4-oxo-1,4-dihydro-[1,10]phenanthroline-3-carboxylic acid (see, e.g., Seki et al. (1974) Chem Abstracts 81:424, No. 21).

Exemplary hydrozones are described in U.S. Pat. No. 6,660,737. All compounds listed in U.S. Pat. No. 6,660,737, in particular, those listed in the compound claims and the final products of the working examples, are hereby incorporated into the present application by reference herein.

Exemplary dihydropyrazoles and dihydropyrozolones are described in U.S. Pat. No. 6,878,729 and International Publication No. WO 2008/049539, respectively. All compounds listed in U.S. Pat. No. 6,878,729, in particular, those listed in the compound claims and the final products of the working examples, are hereby incorporated into the present application by reference herein. Exemplary dipyridyl dihyropyrazones are described in International Publication No. WO 2006/114213. All compounds listed in International Publication No. WO 2006/114213, in particular, those listed in the compound claims and the final products of the working examples, are hereby incorporated into the present application by reference herein.

Exemplary spiroindalones are described in International Publication No. WO 2008/144266. All compounds listed in International Publication No. WO 2008/144266, in particular, those listed in the compound claims and the final products of the working examples, are hereby incorporated into the present application by reference herein.

Additional HIF prolyl hydroxylase inhibitors known to those of skill in the art are described in Dao et al. (2009, Anal Biochem 384(2):213-23), Frohn et al. (2008, Bioorg Med Chem Lett 18(18):5023-6), and Tegley et al. (2008, Bioorg Med Chem Lett 18(14):3925-8). All compounds listed in the foregoing publications are hereby incorporated into the present application by reference herein.

In various embodiments, compounds suitable for use in the present invention are selected from the group consisting of 2-oxoglutarate mimetics, iron chelators, and proline analogs. In preferred embodiments, the compound is a 2-oxoglutarate structural mimetic.

2-oxoglutarate structural mimetics suitable for use in the claimed methods include structural mimetics of 2-oxoglutarate that inhibit HIF prolyl hydroxylase activity competitively with respect to 2-oxoglutarate. In preferred embodiments, the compound is a 2-oxoglutarate structural mimetic that inhibits HIF prolyl hydroxylase competitively with respect to 2-oxoglutarate and noncompetitively with respect to iron.

A compound of the present invention is, in various embodiments, a cyclic carboxamide. In some embodiments, the cyclic carboxamide is a carbonyl glycine. In other embodiments, the carboxamide is replaced by a carbonyl proprionic acid. In some embodiments of the present invention, the compound of the present invention is a carbocyclic carboxamide.

Preferred cyclic carboxamides suitable for use in the present invention are heterocyclic carboxamides. Such heterocyclic carboxamide compounds include heterocyclic carboxamides previously identified as inhibitors of HIF prolyl hydroxylase activity, and known and available to those of skill in the art. In certain embodiments, a compound of the present invention is a heterocyclic carboxamide having a heterocyclic group selected from the group consisting of: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuiranyl. In preferred embodiments, the heterocyclic group is a single ring selected from the group consisting of a pyridine, a pyridinone, a pyradizine, a pyridazinone, a pyrimidine, and a pyrimidinone ring. In other preferred embodiments, the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

A particularly preferred compound of the present invention is selected from the group consisting of an isoquinoline, a pyrrolopyridine, a thiazolopyridine, a quinoline, a pyridinone, and a pyrrolopyridazinone carboxamide.

In a series of embodiments, heterocyclic carboxamides suitable for use in the claimed methods are heterocyclic carbonyl glycines. Preferred such heterocyclic carbonyl glycines include those represented by Formula I, infra.

In successive embodiments, the heterocyclic carbonyl glycine suitable for use in the present invention is a heterocyclic carbonyl glycine having a heterocyclic group that is selected from the following list: azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, thiazole, thiazolidine, thiophene, benzo[b]thiophene, morpholinyl, thiomorpholinyl (also referred to as thiamorpholinyl), piperidinyl, pyrrolidine, and tetrahydrofuranyl. In certain preferred embodiments, the heterocyclic carbonyl glycine suitable for use in the present invention is a heterocyclic carbonyl glycine having a heterocyclic group, wherein the heterocyclic group is a single ring selected from the following list: a pyridine, a pyridinone, a pyradizine, a pyridazinone, a pyrimidine, and a pyrimidinone ring. In other preferred embodiments, the heterocyclic carbonyl glycine suitable for use in the present invention is a heterocyclic carbonyl glycine having a heterocyclic group, wherein the heterocyclic group is a multiple condensed ring selected from the group consisting of an isoquinoline, an isoquinolone, a naphthyridinone, a pyrrolopyridine, a pyrrolopyridinone, a pyrozolopyridinone, a pyrrolopyridizinone, a quinoline, a quinolone, a chromenone, a thiochromenone, a thienopyridine, a thienopyridinone, a thiazolopyridine, and a thiazolopyridinone.

Most preferred heterocyclic carbonyl glycines suitable for use in the claimed methods are heterocyclic carbonyl glycines having a heterocyclic moiety selected from the group consisting of an isoquinoline, a pyrrolopyridine, a thiazolopyridine, a quinoline, a pyridinone, and a pyrrolopyridazinone moiety.

Isoquinoline carbonyl glycines suitable for use in the present invention include isoquinoline-3-carbonyl-glycines; more preferably, 4-hydroxy-isoquinoline-3-carbonyl-glycines. Exemplary 4-hydroxy-isoquinoline-3-carbonyl-glycines include, but are not limited to: [(1-Chloro-4-hydroxy-6-phenylsulfanyl-isoquinoline-3-carbonyl)-amino]-acetic acid (Compound T); [(4-Hydroxy-7-phenoxy-isoquinoline-3-carbonyl)-amino]-acetic acid (Compound B); [(4-Hydroxy-1-methyl-7-phenoxy-isoquinoline-3-carbonyl)-amino]-acetic acid (Compound A); [(1-Benzoyl-4-hydroxy-isoquinoline-3-carbonyl)-amino]-acetic acid (Compound C); (S)-2-[(1-Cyano-4-hydroxy-7-phenoxy-isoquinoline-3-carbonyl)-amino]-propionic acid (Compound G); {[4-Hydroxy-8-(4-methoxy-phenoxy)-1-methyl-isoquinoline-3-carbonyl]-amino}-acetic acid (Compound E); (S)-2-[(1-Cyano4-hydroxy-8-phenoxy-isoquinoline-3-carbonyl)-amino]-propionic acid (Compound F); and [(8-Chloro-4-hydroxy-5-methyl-isoquinoline-3-carbonyl)-amino]-acetic acid (Compound D); and other 4-hydroxy-isoquinoline-3-carbonyl-glycines represented by Formula II, infra.

Preferred pyrrolopyridine carbonyl glycines include pyrrolo[2,3-c]pyridine-5-carbonyl-glycines; in particular, 4-hydroxy-pyrrolo[2,3-c]pyridine-5-carbonyl-glycines. Additionally preferred pyrrolopyridine carbonyl glycines include pyrrolo[3,2-c]pyridine-6-carbonyl-glycines; in particular 7-hydroxy-pyrrolo[3,2-c]pyridine-6-carbonyl-glycines. Exemplary pyrrolopyridine carbonyl glycines include, but are not limited to: {2-[3-Bromo-2-(4-chloro-phenyl)-4-hydroxy-1-phenyl-1H-pyrrolo[2,3-c]pyridine-5-carbonyl)-amino]-acetic acid (Compound H); [(7-Cyano4-hydroxy-1-methoxymethyl-1H-pyrrolo[2,3-c]pyridine-5-carbonyl)-amino]-acetic acid (Compound I); and [(I -Benzyl-2,3-dichloro-7-hydroxy-4-methyl-1H-pyrrolo[3,2-c]pyridine-6-carbonyl)-amino]-acetic acid (Compound U), and other compounds of Formula III, infra.

Other preferred compounds of the present invention are thiazolopyridine carbonyl glycines. Exemplary thiazolopyridine carbonyl glycines include thiazolo[4,5-c]pyridine-6-carboxamides. Particularly preferred thiazolo[4,5-c]pyridine-6-carboxamides include, but are not limited to, 7-hydroxy-thiazolo[4,5-c]pyridine-6-carboxamides. Representative such compounds include (S)-2-[(7-Hydroxy-4-methyl-2-phenyl-thiazolo[4,5-c]pyridine-6-carbonyl)-amino]-propionic acid (Compound S) and [(2-tert-Butyl-7-hydroxy-4-phenethyl-thiazolo[4,5-c]pyridine-6-carbonyl)-amino]-acetic acid (Compound R), and other such compounds encompassed by Formula IV, infra.

Preferred quinoline carbonyl glycines suitable for use in the present methods and medicaments include quinoline-2-carbonyl-glycones, such as [(3-Benzyloxy-7-chloro-quinoline-2-carbonyl)-amino]-acetic acid (Compound P); [(7-Chloro-3-hydroxy-quinoline-2-carbonyl)-amino]-acetic acid (Compound O); and [(3-Hydroxy-6-phenoxy-quinoline-2-carbonyl)-amino]-acetic acid (Compound Q); and other compounds of Formula V, infra.

Also preferred are pyridinone carbonyl glycines; in particular, 2-oxo-1,2-dihydro-pyridine-3-carbonyl-glycines. More preferred are 4-hydroxy-2-oxo-1,2-dihydro-pyridine-3-carbonyl-glycines. Exemplary such compounds are represented by Formula VI, infra, and include, but are not limited to, [(1-Benzyl-4-hydroxy-2-oxo-6-phenyl-1,2-dihydro-pyridine-3-carbonyl)-amino]-acetic acid (Compound J); [(1-Hexyl-4-hydroxy-2-oxo-6-phenyl-1,2-dihydro-pyridine-3-carbonyl)-amino]-acetic acid (Compound K); and (1-Benzyl-4-hydroxy-2-oxo-5-phenyl-1,2-dihydro-pyridine-3-carbonyl)-amino]-acetic acid (Compound L); and other compounds of formula IV, infra.

Other preferred compounds suitable for use in the present invention are pyrrolopyridazinone carbonyl glycines; preferably, 2-oxo-pyrrolo[1,2-b]pyridazine-3-carboxamides; more preferably, 4-hydroxy-2-oxo-pyrrolo[1,2-b]pyridazine-3-carboxamides. Exemplary such compounds include, but are not limited to: [(1-Benzyl-5,6,7-trichloro-4-hydroxy-2-oxo-1,2-dihydro-pyrrolo[1,2-b]pyridazine-3-carbonyl)-amino]-acetic acid (Compound V); {[4-Hydroxy-2-oxo-1-(2-trifluoromethyl-benzyl)-1,2-dihydro-pyrrolo[1,2-b]pyridazine-3-carbonyl]-amino}-acetic acid (Compound M); and [(1-Hexyl-4-hydroxy-2-oxo-1,2-dihydro-pyrrolo[1,2-b]pyridazine-3-carbonyl)-amino]-acetic acid (Compound N); and other compounds of Formula VII, infra.

As discussed, supra, in one embodiment, a compound of the present invention is a HIF prolyl hydroxylase inhibitor compound of Formula I:

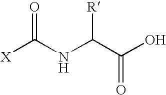

wherein X is an optionally substituted cyclic moiety; and
R′ is hydrogen or (C₁-C₄)-alkyl.

In particular embodiments, the cyclic moiety is a hetercyclic moiety and R′ is hydrogen. Such HIF prolyl hydroyxlase inhibitors include, but are not limited to, variously substituted pyridine-2-carbonyl-glycines, pyridazine-3-carbonyl-glycines, quinoline-2-carbonyl-glycines, 2-oxo-1,2-dihydro-quinoline-3-carbonyl-glycines, 2-oxo-1,2-dihydro-naphthyridine-3-carbonyl-glycines, 6-oxo-4,6-dihydro-pyridopyrazine-7-carbonyl-glycines, isoquinoline-3-carbonyl-glycines, cinnoline-3-carbonyl-glycines, thienopyridine-6-carbonyl-glycines, thienopyridine-5-carbonyl-glycines, thiazolopyridine-6-carbonyl-glycines, thiazolopyridine-5-carbonyl-glycines, hydroxy-pyrrolopyridine-6-carbonyl-glycines, and pyrrolopyridine-5-carbonyl-glycines.

In another embodiment, a compound of the present invention is a compound of Formula II:

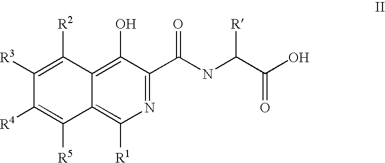

wherein:
R′ is selected from hydrogen and (C₁-C₄)-alkyl;
R¹, R², R³, R⁴ and R⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓC_{f}H_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylanino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido, (C₆-C₁₂)-heteroaryl, (C₇-C₁₆)-heteroaralkyl; where an aryl or heteroaryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂Cl, and —OCF₂—CHFCl;
x is 0 to 3;
f is 1 to 8; and
g is 0 or 1 to (2f+1);
or a pharmaceutically acceptable salt, single stereoisomer, mixture of stereoisomers, ester, or prodrug thereof.

In one embodiment, the compound of the present invention is a compound of Formula II wherein:
R′ is hydrogen or (C₁-C₃)-alkyl;
R¹ is selected from hydrogen, halo, (C₁-C₃)-alkyl, cyano, or arylacyl;
R² is hydrogen or (C₁-C₃)-alkyl;
R³ is hydrogen or arylsulfanyl;
R⁴ is hydrogen or aryloxy; and
R⁵ is selected from hydrogen, halo, aryloxy, or aryloxy substituted with (C₁-C₃)-alkoxy.

In another embodiment, a compound of the present invention is a compound of Formula II wherein:
R′ is hydrogen or methyl;
R¹ is selected from hydrogen, chloro, methyl, cyano, or benzoyl;
R² is selected from hydrogen or methyl;
R³ is hydrogen or phenylsulfanyl;
R⁴ is selected from hydrogen or phenoxy; and
R⁵ is selected from hydrogen, chloro, phenoxy, or 4-methoxy-phenoxy.

In various embodiments, a compound of the present invention is a compound of Formula III:

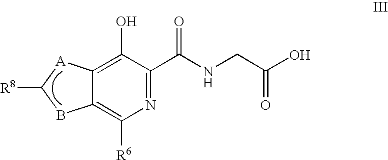

wherein:

one of A or B is ═C(R⁷)— and the other is —N(R⁹)—;

[symbol] independently represents a single or a double bond;
R⁶ is selected from the group consisting of hydrogen, halo, cyano, (C₁-C₃)-alkyl, and aryl;
R⁷ is selected from the group consisting of hydrogen, halo, cyano, (C₁-C₆)-alkyl, and aryl, wherein the aryl is optionally substituted by one or two halo;
   R⁸ is selected from the group consisting of hydrogen, halo, cyano, (C₁-C₆)-alkyl, trifluoromethyl, and aryl optionally substituted with halo; and
R⁹ is selected from the group consisting of hydrogen, (C₁-C₁₀)-alkyl, (C₁-C₃)-alkyl-(C₁-C₁₀)-alkyl, (C₁-C₃)-alkoxy-(C₁-C₆)-alkyl, (C₄-C₆)-cycloalkyl, (C₄-C₆)-cycloalkyl-(C₁-C₃)-alkyl, aryl, (C₇-C₁₂)-aralkyl, aryl-aralkyl, and heteroaralkyl; where in each case an aryl or heteroaryl may be optionally substituted by one or two halo, trifluoromethyl, or (C₁-C₄)-alkoxy;
or pharmaceutically acceptable salts, single stereoisomers, mixtures of stereoisomers, esters, or prodrugs thereof.

In one embodiment, a compound of the present invention is a compound of Formula III wherein:
A is ═C(R⁷)—;
B is —N(R⁹)—;
R⁶ is selected from hydrogen or cyano;
R⁷ is selected from hydrogen or halo;
R⁸ is selected from hydrogen or aryl optionally substituted with halo; and
R⁹ is selected from (C₁-C₃)-alkoxy-(C₁-C₃)-alkyl.

In a further embodiment, a compound of the present invention is a compound of Formula III wherein:
A is ═C(R⁷)—;
B is —N(R⁹)—;
R⁶ is selected from hydrogen or cyano;
R⁷ is selected from hydrogen or bromo;
R⁸ is selected from hydrogen or 4-chloro-phenyl; and
R⁹ is selected from methoxymethyl or phenyl.

In yet another embodiment of the present invention, a compound suitable for use in the claimed methods and medicaments is a compound of Formula III wherein:
A is —N(R⁹)—;
B is ═C(R⁷)—;
R⁶ is (C₁-C₃)-alkyl;
R⁷ is halogen;
R⁸ is halogen; and
R⁹ is aralkoxy.

In another embodiment, a compound of the present invention is a compound of Formula III wherein:
R⁶ is methyl;
R⁷ is chloro;
R⁸ is chloro; and
R⁹ is benzyl.

It is contemplated in some embodiments of the present invention that a compound of the invention is a compound represented by Formula IV,

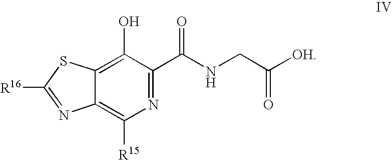

wherein:
R¹⁵ is selected from the group consisting of hydrogen, cyano, acetyl, (C₁-C₆)-alkyl, (C₁-C₄)-alkynyl, (C₇-C₁₂)-aralkyl, (C₇-C₁₂)-aralkenyl, (C₁-C₆)-alkyl-sulfanyl-(C₁-C₃)-alkyl, aryl, heterocyclyl, and heteroaryl; wherein each substituent is optionally substituted with halo or cyano; and
R¹⁶ is selected from the group consisting of (C₁-C₆)-alkyl, (C₆-C₂₂)-aryl optionally substituted with one or two substituents each independently selected from cyano, (C₁-C₄)-alkyl, trifluoromethyl, halo, (C₁-C₄)-alkoxy, aryl, or aryloxy; aryloxy, heteocyclyl, and heteroaryl optionally substituted with halo, (C₁-C₄)-alkoxy, aryloxy, or arylsulfanyl;
or pharmaceutically acceptable salts, single stereoisomers, mixtures of stereoisomers, esters, or prodrugs thereof

In certain embodiments, a compound of the present invention is a compound of Formula IV wherein:
R¹⁵ is (C₁-C₃)-alkyl or (C₇-C₁₂)-aralkyl; and
R¹⁶ is selected from (C₁-C₆)-alkyl or (C₆-C₁₂)-aryl.

In other embodiments, a compound of the present invention is a compound of Formula IV, wherein:
R¹⁵ is selected from methyl or phenethyl; and
R¹⁶ is selected from t-butyl or phenyl.

It is also contemplated that in certain embodiments a compound suitable for use in the present methods and medicaments is a compound of Formula V:

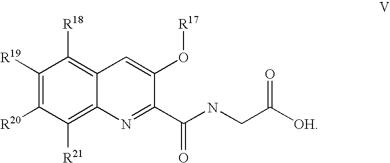

wherein:
R¹⁷ is is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
R¹⁸, R¹⁹, R²⁰, and R²¹ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido, (C₆-C₁₂)-heteroaryl, (C₇-C₁₆)-heteroaralkyl; where an aryl or heteroaryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓC_{f}H_{(2f+1-g)}F_{g}, —OCF₂CI, and —OCF₂—CHFCl;
x is 0 to 3;
f is 1 to 8; and
g is 0 or 1 to (2f+1);
or a pharmaceutically acceptable salt, single stereoisomer, mixture of stereoisomers, ester, or prodrug thereof.

In a further embodiment, a compound of the present invention is a compound of Formula V wherein:
R¹⁷ is selected from hydrogen or aralkoxy;
R¹⁸ and R²¹ are hydrogen;
R¹⁹ is selected from hydrogen or aryloxy; and
R²⁰ is selected from hydrogen or halo.

In another embodiment, a compound of the present invention is a compound of Formula V wherein:
R¹⁷ is selected from hydrogen or benzyloxy;
R¹⁸ and R²¹ are hydrogen;
R¹⁹ is selected from hydrogen or phenoxy; and
R²⁰ is selected from hydrogen or chloro.

It is contemplated that a compound of the present invention is in various embodiments a compound of Formula VI:

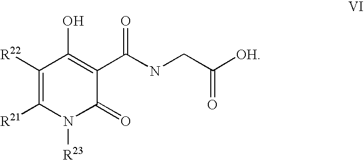

wherein:
R²¹ is selected from hydrogen or aryl;
R²² is selected from hydrogen or aryl; and
R²³ is selected from (C₁-C₁₀)-alkyl or aralkyl.

In one embodiment, the compound of the present invention is a compound of Formula VI wherein:
R²¹ is selected from hydrogen or phenyl;
R²² is selected from hydrogen or phenyl; and
R²³ is selected from hexyl or benzyl.

In another embodiment, a compound for use in the present methods and medicaments is a compound of Formula VII:

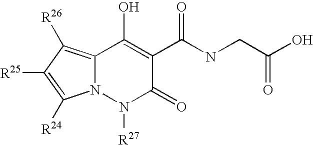

wherein:
R²⁴, R²⁵, and R²⁶ are each independently selected from hydrogen or halogen; and
R²⁷ is (C₇-C₁₂)-aralkyl optionally substituted on the aryl with one or two substituents selected from the group consisting of halo, trifluoromethyl, and (C₁-C₃)-alkoxy.

In certain embodiments, the compound of the present invention is a compound of Formula VII wherein:
R²⁴ is selected from hydrogen or halo;
R²⁵ is selected from hydrogen or halo;
R²⁶ is selected from hydrogen or halo; and
R²⁷ is selected from (C₁-C₁₀)-alkyl or (C₇-C₁₂)-aralkyl, wherein the aryl is optionally substituted with trifluoromethyl.

In further embodiments, the compound of the present invention is a compound of Formula VII wherein:
R²⁴ is selected from hydrogen or chloro;
R²⁵ is selected from hydrogen or chloro;
R²⁶ is selected from hydrogen or chloro; and
R²⁷ is selected from hexyl, benzyl, or 2-trifluoromethyl-benzyl.

The terms “hydroxy” or “hydroxyl” refer to the group —H.

The term “halo” or “halogen” refers to fluoro, chloro, bromo, and iodo.

The term “cyano” refers to the group —CN.

The term “nitro” refers to the group —NO₂.

The term “carboxyl” refers to —COOH or salts thereof.

The term “alkyl” refers to saturated monovalent hydrocarbyl groups having from 1 to 10 carbon atoms; more particularly, from 1 to 5 carbon atoms; and, even more particularly, 1 to 3 carbon atoms. This term is exemplified by groups such as methyl, ethyl, n-propyl, iso-propyl, n-butyl, t-butyl, n-pentyl, and the like.

The term “cycloalkyl” refers to a saturated or an unsaturated, but nonaromatic, cyclic alkyl groups of from 3 to 10, 3 to 8, or 3 to 6 carbon atoms having single or multiple cyclic rings including, by way of example, adamantyl, cyclopropyl, cyclobutyl, cyclopentyl, cyclooctyl, cyclohexenyl, and the like.

The term “cycloalkoxy” refers to an —O-cycloalkyl group.

The term “aryl” refers to a monovalent aromatic carbocyclic group of from 6 to 14 carbon atoms having a single ring (e.g., phenyl) or multiple condensed rings (e.g., naphthyl or anthryl), which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

The terms “heterocyclic” or “heterocyclyl” refer to a saturated or unsaturated ring system having a single ring or multiple condensed rings, from 1 to 10 carbon atoms, and from 1 to 4 hetero atoms selected from the group consisting of nitrogen, sulfur, or oxygen within the ring.

The term “heteroaryl” refers to an aromatic heterocyclic group of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, or thienyl) or multiple condensed rings (e.g., indolizinyl or benzothienyl), which condensed rings may or may not be aromatic provided the point of attachment is through a ring containing the heteroatom and that ring is aromatic. The nitrogen can optionally be oxidized to provide for the N-oxide, and/or the sulfur ring atoms can optionally be oxidized to provide for the sulfoxide and sulfone derivatives.

Examples of heterocycles and heteroaryls include, but are not limited to, azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

The term “alkenyl” refers to a vinyl unsaturated monovalent hydrocarbyl group having from 2 to 6, preferably from 2 to 4, carbon atoms, and having at least 1, preferably from 1 to 2, sites of vinyl (>C═C<) unsaturation. Such groups are exemplified by vinyl (ethen-1-yl), allyl, but-3-enyl, and the like.

The term “alkynyl” refers to acetylinic unsaturated monovalent hydrocarbyl groups having from 2 to 6, preferably from 2 to 3, carbon atoms and having at least 1, preferably from 1 to 2, sites of acetylenic (—C≡C—) unsaturation. This group is exemplified by ethyn-1-yl, propyn-1-yl, propyn-2-yl, and the like.

The term “alkoxy” refers to the group “alkyl-O—,” which includes, by way of example, methoxy, ethoxy, n-propoxy, iso-propoxy, n-butoxy, t-butoxy, sec-butoxy, n-pentoxy, and the like.

The term “alkenyloxy” refers to the group “alkenyl-O—.”

The term “alkynyloxy” refers to the group “alkynyl-O—.”

The term “aryloxy” refers to the group aryl-O— that includes, by way of example, phenoxy, naphthoxy, and the like.

The term “aralkyloxy” refers to the group aralkyl-O— that includes, by way of example, benzyloxy, and the like.

The term “carbonyl” refers to C═O.

The term “carbonyloxy” refers to —C(═O)O—.

The terms “aminoacyl” or “amide”, or the prefixes “carbamoyl” or “carboxamide,” refer to the group —C(O)NR^{q}R^{q} where each R^{q} is independently selected from the group consisting of hydrogen, alkyl, alkenyl, alkynyl, aryl, cycloalkyl, heteroaryl, and heterocyclic; or where each R^{q} is joined to form together with the nitrogen atom a heterocyclic wherein alkyl, alkenyl, alkynyl, cycloalkyl, aryl, heteroaryl, and heterocyclic are as defined herein.

The term “amino” refers to the group —NH₂.

The terms “thio” or “mercapto” refer to the group —SH.

The terms “alkylsulfanyl,” “alkylthio,” or “thioether” refer to the groups -S-alkyl where alkyl is as defined above.

The term “sulfinyl” refers to the group —S(O)—.

The term “sulfonyl” refers to the group —S(O)₂—.

The term “heterocyclyloxy” refers to the group —O-heterocyclic.

The term “cycloalkylene” refers to divalent cycloalkyl groups as defined above. The terms “cycloalkylthio” or “cycloalkylsulfanyl” refer to the groups —S-cycloalkyl where cycloalkyl is as defined herein.

The terms “arylthio” or “arylsulfanyl” refer to the group —S-aryl, where aryl is as defined herein.

The terms “heteroarylthio” or “heteroarylsulfanyl” refer to the group —S-heteroaryl, where heteroaryl is as defined herein.

The terms “heterocyclicthio” or “heterocyclicsulfanyl” refer to the group —S-heterocyclic, where heterocyclic is as defined herein.

The term “alkyl alcohol” refers to the group “alkyl-OH”. “Alkyl alcohol” is meant to include methanol, ethanol, 2-propanol, 2-butanol, butanol, etc.

The term “acyl” refers to the groups H-C(O)—, alkyl-C(O)—, alkenyl-C(O)—, alkynyl-C(O)—, cycloalkyl-C(O)—, aryl-C(O)—, heteroaryl-C(O)—, and heterocyclic-C(O)—, provided that a nitrogen atom of the heterocyclic is not bound to the —C(O)— group, wherein alkyl, alkenyl, alkynyl, cycloalkyl, aryl, heteroaryl, and heterocyclic are as defined herein.

The term “acyloxy” refers to the groups alkyl-C(O)O—, alkenyl-C(O)O—, alkynyl-C(O)O—, aryl-C(O)O—, cycloalkyl-C(O)O—, heteroaryl-C(O)O—, and heterocyclic-C(O)O—, wherein alkyl, alkenyl, alkynyl, cycloalkyl, aryl, heteroaryl, and heterocyclic are as defined herein.

The term “alkenyl” refers to a vinyl unsaturated monovalent hydrocarbyl group having from 2 to 6 carbon atoms, and preferably 2 to 4 carbon atoms, and having at least 1, and preferably from 1 to 2 sites of vinyl (>C═C<) unsaturation. Such groups are exemplified by vinyl (ethen-1-yl), allyl, but-3-enyl and the like.

The term “alkynyl” refers to acetylinic unsaturated monovalent hydrocarbyl groups having from 2 to 6, preferably from 2 to 3, carbon atoms and having at least 1, preferably from 1 to 2, sites of acetylenic (—C≡C—) unsaturation. This group is exemplified by ethyn-1-yl, propyn-1-yl, propyn-2-yl, and the like.

The term “acylamino” refers to the groups —NR^{t}C(O)alkyl, —NR^{t}C(O)cycloalkyl, —NR^{t}C(O)alkenyl, —NR^{t}C(O)alkynyl, —NR^{t}C(O)aryl, —NR^{t}C(O)heteroaryl, and -NR^{t}C(O)heterocyclic where R^{t} is hydrogen or alkyl, and wherein alkyl, alkenyl, alkynyl, cycloalkyl, aryl, heteroaryl, and heterocyclic are defined herein.

The term “carbonyloxyamino” refers to the groups —NR^{u}C(O)O-alkyl, —NR^{u}C(O)O-alkenyl, —NR^{u}C(O)O-alkynyl, —NR^{u}C(O)O-cycloalkyl, —NR^{u}C(O)O-aryl, —NR^{u}C(O)O-heteroaryl, and —NR^{u}C(O)O-heterocyclic, where R^{u} is hydrogen or alkyl and wherein alkyl, alkenyl, alkynyl, cycloalkyl, aryl, heteroaryl, and heterocyclic are as defined herein.

The term “oxycarbonylamino” refers to the groups —NR^{u}C(O)O-alkyl, —NR^{u}C(O)O-alkenyl, —NR^{u}C(O)O-alkynyl, —NR^{u}C(O)O-cycloalkyl, —NR^{u}C(O)O-aryl, —NR^{u}C(O)O-heteroaryl, and —NR^{u}C(O)O-heterocyclic, where R^{u} is hydrogen or alkyl, and wherein alkyl, alkenyl, alkynyl, cycloalkyl, aryl, heteroaryl, and heterocyclic are as defined herein.

The term “oxythiocarbonylamino” refers to the groups —NR^{u}C(S)O-alkyl, —NR^{u}C(S)O-alkenyl, —NR^{u}C(S)O-alkynyl, —NR^{u}C(S)O-cycloalkyl, —NR^{u}C(S)O-aryl, —NR^{u}C(S)O-heteroaryl, and —NR^{u}C(S)O-heterocyclic, where R^{u} is hydrogen or alkyl, and wherein alkyl, alkenyl, alkynyl, cycloalkyl, aryl, heteroaryl, and heterocyclic are as defined herein.

The term “aminocarbonyloxy” or the prefix “carbamoyloxy” refer to the groups —OC(O)NR^{v}R^{v} where each R^{v} is independently selected from the group consisting of hydrogen, alkyl, alkenyl, alkynyl, cycloalkyl, aryl, heteroaryl, and heterocyclic; or where each R^{v} is joined to form, together with the nitrogen atom, a heterocyclic, and wherein alkyl, alkenyl, alkynyl, cycloalkyl, aryl, heteroaryl, substituted heteroaryl, and heterocyclic are as defined herein.

The term “aminocarbonylamino” refers to the group —NR^{w}C(O)N(R^{w})₂ where each R^{w} is independently selected from the group consisting of hydrogen and alkyl.

The term “aminothiocarbonylamino” refers to the group —NR^{w}C(S)N(R^{w})₂ where each R^{w} is independently selected from the group consisting of hydrogen and alkyl.

The term “aryloxyaryl” refers to the group -aryl-O-aryl.

The term “carboxyl ester” refers to the groups —C(O)O-alkyl, —C(O)O-alkenyl, —C(O)O-alkynyl, —C(O)O-cycloalkyl, —C(O)O-aryl, —C(O)O-substituted aryl, —C(O)O-heteroaryl, —C(O)O-substituted heeteroaryl, —C(O)O-heterocyclic, and —C(O)O-substituted heterocyclic.

The term “cycloalkylene” refers to divalent cycloalkyl groups as defined above.

The term “heteroaryloxy” refers to the group —O-heteroaryl.

The term “sulfonyl” refers to the group —S(O)₂—, and may be included in the groups —S(O)₂H, —SO₂-alkyl, —SO₂-alkenyl, —SO₂-alkynyl, —SO₂-cycloalkyl, —SO₂-cycloalkenyl, —SO₂-aryl, —SO₂-substituted aryl, —SO₂-heteroaryl, and —SO₂-heterocyclic, wherein alkyl, alkenyl, alkynyl, cycloalkyl, cycloalkenyl, aryl, heteroaryl, and heterocyclic are as defined herein.

The term “heterocyclyloxy” refers to the group —O-heterocyclic.

The terms “arylthio” or “arylsulfanyl” refer to the group —S-aryl.

The terms “heteroarylthio” or “heteroarylsulfanyl” refer to the group —S-heteroaryl.

The terms “heterocyclicthio” or “heterocyclicsulfanyl” refer to the group —S-heterocyclic.

Conjugated terms refer to a linear arrangement of the separate substituents as each separate term is defined herein. For example, the term “aralkyl” refers to an aryl-alkyl group and includes, by way of example, benzyl; the term “aralkylcarbamoyl” refers to an aryl-alkyl-carbomoyl substituent wherein each term is as defined herein, etc.

It is understood that in all substituted and conjugated groups as defined herein, polymers arrived at by defining substituents with further substituents to themselves (e.g., aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three.

Similarly, it is understood that the above definitions are not intended to include impermissible substitution patterns (e.g., methyl substituted with 5 fluoro groups or a hydroxyl group alpha to ethenylic or acetylenic unsaturation). Such impermissible substitution patterns are well known to the skilled artisan.

The term “pharmaceutically acceptable salt” refers to pharmaceutically acceptable salts of a compound, which salts are derived from a variety of organic and inorganic counter ions well known in the art, and include, by way of example only, sodium, potassium, calcium, magnesium, ammonium, tetraalkylammonium, and the like; and, when the molecule contains a basic functionality, salts of organic or inorganic acids, such as hydrochloride, hydrobromide, tartrate, mesylate, acetate, maleate, oxalate, and the like.

The terms “stereoisomer” or “stereoisomers” refer to compounds that differ in the chirality of one or more stereocenters. Stereoisomers include enantiomers (compounds are non-superimposable mirror images) and diastereomers (compounds having more than one stereogenic center that are non-mirror images of each other and wherein one or more stereogenic center differs between the two stereoisomers). The compounds of the invention can be present as a mixture of stereoisomers or as a single stereoisomer.

The term “tautomer” refers to alternate forms of a compound that differ in the position of a proton, such as enol, keto, and imine enamine tautomers, or the tautomeric forms of heteroaryl groups contining a ring atom attached to both a ring NH moiety and a ring ═N moiety such as pyrazoles, imidazoles, benzimidazoles, triazoles, and tetrazoles.

The term “prodrug,” as used herein, refers to compounds that include chemical groups which, in vivo, can be converted into the carboxylate group and/or can be split off from the amide N-atom and/or can be split off from the R′ atom to provide for the active drug, a pharmaceutically acceptable salt thereof, or a biologically active metabolite thereof. Suitable groups are well known in the art and particularly include: for the carboxylic acid moiety, a prodrug selected from, e.g., esters including, but not limited to, those derived from alkyl alcohols, substituted alkyl alcohols, hydroxy substituted aryls and heteroaryls and the like; amides, particularly amides derived from amines of the Formula HNR²⁰⁰R²¹⁰ where R²⁰⁰ and R²¹⁰ are independently hydrogen, alkyl, substituted alkyl, aryl, substituted aryl, and the like; hydroxymethyl, aldehyde and derivatives thereof. The term “ester” refers to compounds that include the group —COOR where R is alkyl, substituted alkyl, alkoxy, or substituted alkoxy.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Methods for Identifying Compounds

Methods for identifying compounds of the invention are also provided. Assays for hydroxylase activity are standard in the art. Such assays can directly or indirectly measure hydroxylase activity. For example, an assay can measure hydroxylated residues, e.g., proline, asparagine, etc., present in the enzyme substrate, e.g., a target protein, a synthetic peptide mimetic, or a fragment thereof. (See, e.g., Palmerini et al. (1985) J Chromatogr 339:285-292.) A reduction in hydroxylated residue, e.g., proline or asparagine, in the presence of a compound is indicative of a compound that inhibits hydroxylase activity. Alternatively, assays can measure other products of the hydroxylation reaction, e.g., formation of succinate from 2-oxoglutarate. (See, e.g., Cunliffe et al. (1986) Biochem J 240:617-619.) Kaule and Gunzler (1990; Anal Biochem 184:291-297) describe an exemplary procedure that measures production of succinate from 2-oxoglutarate.

Procedures such as those described above can be used to identify compounds that modulate HIF hydroxylase activity. Target protein may include HIFα or a fragment thereof, e.g., HIF(556-575). Enzyme may include, e.g., HIF prolyl hydroxylase or active fragments thereof (see, e.g., GenBank Accession No. AAG33965, etc.) or HIF asparaginyl hydroxylase or active fragments thereof(see, e.g., GenBank Accession No. AAL27308, etc.), obtained from any source. Enzyme may also be present in a crude cell lysate or in a partially purified form. For example, procedures that measure HIF hydroxylase activity are described in Ivan et al. (2001, Science 292:464-468; and 2002, Proc Natl Acad Sci USA 99:13459-13464) and Hirsila et al. (2003, J Biol Chem 278:30772-30780); additional methods are described in International Publication No. WO 03/049686. Measuring and comparing enzyme activity in the absence and presence of the compound will identify compounds that inhibit hydroxylation of HIFα.

### Pharmaceutical Formulations and Routes of Administration

The compositions of the present invention can be delivered directly or in pharmaceutical compositions containing excipients, as is well known in the art. The present methods of treatment involve administration of an effective amount of a compound of the present invention to a subject in need, wherein the subject would benefit from inhibition of the differentiation of a T helper cell into a Th1 cell.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracistemal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In preferred embodiments, the compounds of the present invention are administered orally. For example, in certain embodiments, the invention provides for oral administration of [(4-Hydroxy-1-methyl-7-phenoxy-isoquinoline-3-carbonyl)-amino]-acetic acid (Compound A), [(4-Hydroxy-7-phenoxy-isoquinoline-3-carbonyl)-amino]-acetic acid (Compound B), [(1-Benzoyl-4-hydroxy-isoquinoline-3-carbonyl)-amino]-acetic acid (Compound C), [(8-Chloro-4-hydroxy-5-methyl-isoquinoline-3-carbonyl)-amino]-acetic acid (Compound D), {[4-Hydroxy-8-(4-methoxy-phenoxy)-1-methyl-isoquinoline-3-carbonyl]-amino}-acetic acid (Compound E), (S)-2-[(1-Cyano4-hydroxy-8-phenoxy-isoquinoline-3-carbonyl)-amino]-propionic acid (Compound F), (S)-2-[(1-Cyano4-hydroxy-7-phenoxy-isoquinoline-3-carbonyl)-amino]-propionic acid (Compound G), {2-[3-Bromo-2-(4-chloro-phenyl)-4-hydroxy-1-phenyl-1H-pyrrolo[2,3-c]pyridine-5-carbonyl)-amino]-acetic acid (Compound H), [(7-Cyano-4-hydroxy-1-methoxymethyl-1H-pyrrolo[2,3-c]pyridine-5-carbonyl)-amino]-acetic acid (Compound I), [(1-Benzyl-4-hydroxy-2-oxo-6-phenyl-1,2-dihydro-pyridine-3-carbonyl)-amino]-acetic acid (Compound J), [(1-Hexyl-4-hydroxy-2-oxo-6-phenyl-1,2-dihydro-pyridine-3-carbonyl)-amino]-acetic acid (Compound K), [(1-Benzyl-4-hydroxy-2-oxo-5-phenyl-1,2-dihydro-pyridine-3-carbonyl)-amino]-acetic acid (Compound L), {[4-Hydroxy-2-oxo-1-(2-trifluoromethyl-benzyl)-1,2-dihydro-pyrrolo[1,2-b]pyridazine-3-carbonyl]-amino}-acetic acid (Compound M), [(1-Hexyl-4-hydroxy-2-oxo-1,2-dihydro-pyrrolo[1,2-b]pyridazin-3-carbonyl)-amino]-acetic acid (Compound N), [(7-Chloro-3-hydroxy-quinoline-2-carbonyl)-amino]-acetic acid (Compound O), [(3-Benzyloxy-7-chloro-quinoline-2-carbonyl)-amino]-acetic acid (Compound P), [(3-Hydroxy-6-phenbxy-quinoline-2-carbonyl)-amino]-acetic acid (Compound Q), [(2-tert-Butyl-7-hydroxy-4-phenethyl-thiazolo[4,5-c]pyridine-6-carbonyl)-amino]-acetic acid (Compound R), (S)-2-[(7-Hydroxy-4-methyl-2-phenyl-thiazolo[4,5-c]pyridine-6-carbonyl)-amino]-propionic acid (Compound S), [(1-Chloro-4-hydroxy-6-phenylsulfanyl-isoquinoline-3-carbonyl)-amino]-acetic acid (Compound T), [(1-Benzyl-2,3-dichloro-7-hydroxy4-methyl-1H-pyrrolo[3,2-c]pyridine-6-carbonyl)-amino]-acetic acid (Compound U), or [(1-Benzyl-5,6,7-trichloro-4-hydroxy-2-oxo-1,2-dihydro-pyrrolo[1,2-b]pyridazine-3-carbonyl)-amino]-acetic acid (Compound V).

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal, or human that is being sought by the researcher, veterinarian, medical doctor, or other clinician, e.g., treatment of cancer, including induction of anti-tumor effects, etc.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiments of the present invention, effective doses for compounds of the invention include doses of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, and 30 mg/kg, respectively.

In additional embodiments, effective treatment regimes for compounds of the invention include administration two or three times weekly.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Decreased Secretion of Pro-Inflammatory Cytokines in T Helper Cells

The effect of methods and compounds of the present invention on T helper cell secretion of pro-inflammatory cytokines was evaluated as follows. Naive T helper cells (AllCells LLC, Emeryville, Calif.) obtained from human cord blood were stimulated (i.e., T cell receptor (TCR) stimulation) by seeding into 96-well culture dishes coated with 5 μg/ml anti-CD3 antibody (clone UCHT1, R&D Systems, Minneapolis, Minn.) (Tanaka et.al. (1989) J Immunol 142:2791-2795.), and 5 μg/ml anti-CD28 antibody (clone ANC28.1/D10, Ancell Corporation, Bayport, Minn.), and cultured at 37° C., 5% CO₂ in RPMI-1640 (Mediatech Inc., Herndon, Va.) containing 1% FBS, 1 U/ml penicillin-1 μg/ml streptomycin (Invitrogen Corporation, Carlsbad, Calif.), and 50 U/ml IL-2 (Roche Diagnostics Corporation, Indianapolis, Ind.). At the time of seeding, cells were cultured with media alone or with media containing 2 ng/ml IL-12 (R&D Systems, Minneapolis, Minn.) (to stimulate differentiation of the T helper cells into Th1 cells) and 10 μg/ml anti-IL4 antibody (R&D Systems, Minneapolis, Minn.) (to block differentiation of the T helper cells into Th2 cells). Compound A or 1% DMSO (Veh) was added to the existing media and the cells were cultured for an additional seven days.

The conditioned media was then collected from the cell cultures and analyzed for interferon-gamma (IFN-γ) and tumor necrosis factor-alpha (TNF-α) levels using MS2400 Human Tissue Culture Cytokine Detection Kits (MesoScale Technologies, Gaithersburg, Md.) according to the manufacturer's instructions.

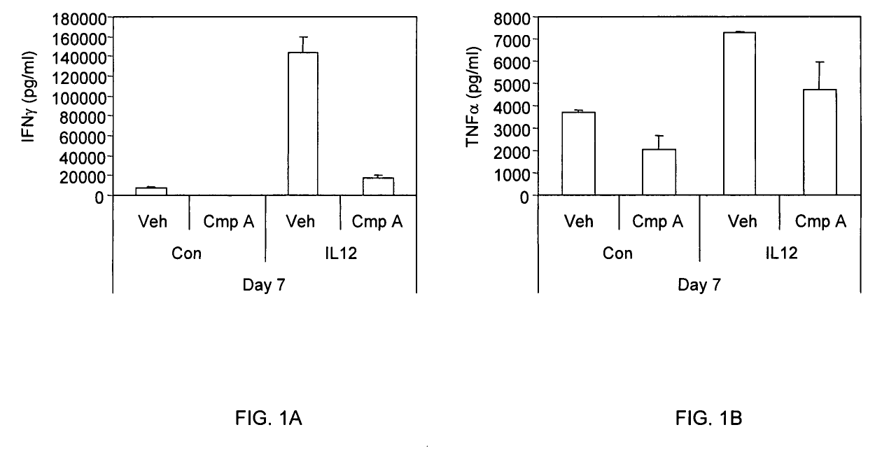

As shown in FIGS. 1A and 1B, treatment of TCR-stimulated T helper cells with Compound A (without IL-12 addition) resulted in a reduction of IFN-γ and TNF-α levels in the conditioned media of the cells, respectively. TCR-stimulated T helper cells treated with IL-12 in the absence of Compound A showed increased IFN-γ levels and increased TNF-α levels compared to the IFN-γ levels and TNF-α levels observed in vehicle-only control treated cells. As IL-12 stimulates the differentiation of T helper cells into Th1 cells, evidenced by the induction of IFN-γ and TNF-α synthesis, these results indicated that the TCR-stimulated T helper cells differentiated into Th1 cells in response to IL-12 addition. Addition of IL-12 to the T helper cells in the presence of Compound A inhibited the increase in IFN-γ and TNF-α levels compared to that observed in cells treated with IL-12 and vehicle in the absence of Compound A. These results indicated that Compound A inhibited IL-12 activity and inhibited differentiation of the TCR-stimulated T helper cells into Th1 cells, and thus showed that methods and compounds of the present invention are useful for decreasing secretion of pro-inflammatory cytokines (IFN-γ and TNF-α) from T helper cells. These results further demonstrated that methods and compounds of the present invention are useful for inhibiting IL-12-mediated differentiation of T helper cells into Th1 cells.

In another series of experiments, the effect of methods and compounds of the present invention on T helper cell secretion of pro-inflammatory cytokines was evaluated as follows. Na\dot{i}ve T helper cells were stimulated substantially as described above with the following modification: cell cultures contained 100 U/ml penicillin-100 U/ml streptomycin. Various compounds of the present invention or 1% DMSO (Veh) was added to the existing media and the cells were cultured for an additional six days.

Following stimulation with IL-12, conditioned media was then collected from the cell cultures and analyzed for interferon-gamma (IFN-γ) levels using MS2400 Human Tissue Culture Cytokine Detection Kits (MesoScale Technologies, Gaithersburg, Md.) according to the manufacturer's instructions.

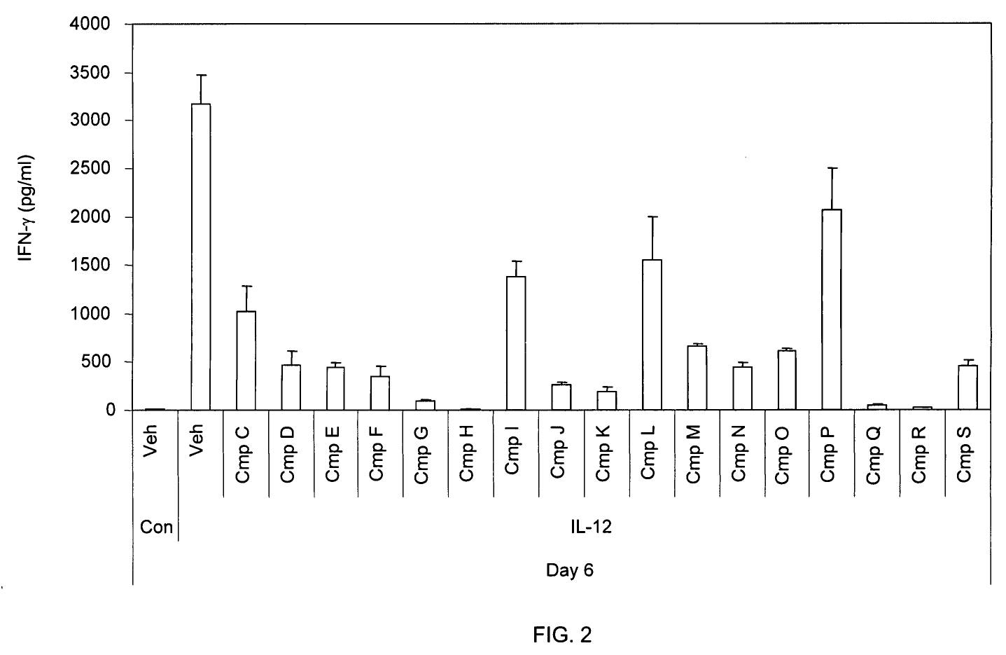

As shown in FIG. 2, TCR-stimulated T cells treated with IL-12 in the absence of compound showed increased IFN-γ levels compared to the IFN-γ levels observed in vehicle-only control TCR-stimulated T cells. As IL-12 stimulates the differentiation of na\dot{i}ve T cells into Th1 cells, evidenced by the induction of IFN-γ synthesis, these results indicated that the TCR-stimulated T cells differentiated into Th1 cells in response to IL-12 addition. Addition of compounds of the invention to IL-12 treated, TCR-stimulated T cells inhibited the increase in IFN-γ levels compared to that observed in TCR-stimulated T cells treated with IL-12 in the absence of compounds of the invention (See FIG. 2). These results indicated that the compounds of the invention inhibited IL-12 activity and inhibited IL-12 mediated Th1 differentiation, and thus showed that methods and compounds of the present invention are useful for decreasing secretion of IL-12 induced IFN-γ from T cells and for inhibiting IL-12-mediated differentiation of T cells into Th1 cells.

### Example 2

### No Repression of T Helper Cell Number or Viability

To examine the effect of compounds and methods of the present invention on T helper cell number and viability, the following studies were performed. Human cord blood na\dot{i}ve T helper cells (AllCells LLC, Emeryville, Calif.) were stimulated (i.e., T cell receptor (TCR) stimulation) by seeding into 96-well culture dishes coated with 5 μg/ml anti-CD3 antibody (clone UCHT1, R&D Systems, Minneapolis, Minn.), and 5 μg/ml anti-CD28 antibody (clone ANC28.1/D10, Ancell Corporation, Bayport, Minn.), and cultured at 37° C, 5% CO₂ in RPMI-1640 (Mediatech Inc., Herndon, Va.) containing 1% FBS, 1 U/ml penicillin-1 μg/ml streptomycin (invitrogen Corporation, Carlsbad, Calif.), and 50 U/ml IL-2 (Roche Diagnostics Corporation, Indianapolis, Ind.). At time of seeding, the cells were cultured with media alone or with media containing 2 ng/ml IL-12 (R&D Systems, Minneapolis, Minn.) (to stimulate differentiation of the T helper cells into Th1 cells) and 10 μg/ml anti-WL4 antibody (R&D Systems, Minneapolis, Minn.) (to block differentiation of the T helper cells into Th2 cells). Compound B or 1% DMSO (Veh) was added to the culture media and the cells were cultured for an additional seven days. The T helper cells were then collected and cell number and cell viability were analyzed using Guava ViaCount reagents and the Guava PCA System (Guava Technologies, Hayward, Calif.).

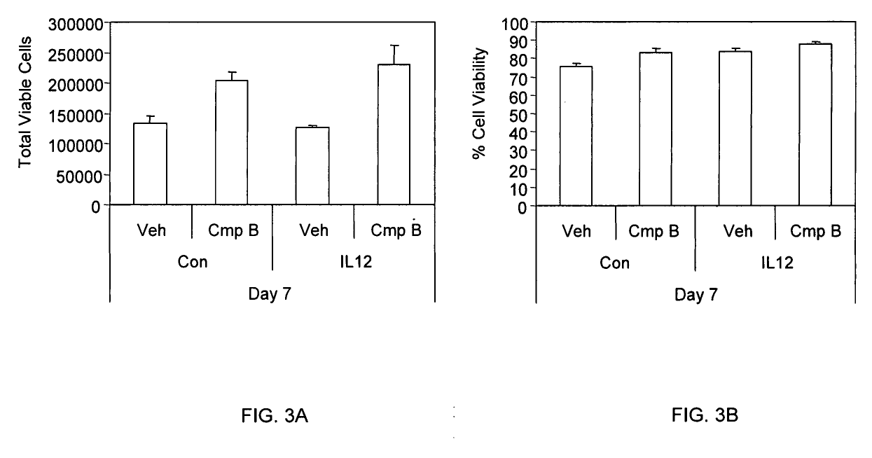

As shown in FIGS. 3A and 3B, T helper cell viability was not reduced by addition of Compound A. Specifically, Compound A did not decrease the total number or the percent of viable T helper cells present at day 7 in these studies. These results showed that methods and compounds of the present invention do not decrease T helper cell number or compromise viability.

In another set of experiments, the effect of compounds and methods of the present invention on T helper cell number and viability was determined. Na\dot{i}ve T helper cells were stimulated substantially as described above with the following modification: cell cultures contained 100 U/ml penicillin-100 U/ml streptomycin. Various compounds of the present invention or 1% DMSO (Veh) was added to the existing media and the cells were cultured for an additional six days. The T helper cells were then collected and cell number and cell viability were analyzed using Guava ViaCount reagents and the Guava PCA System (Guava Technologies, Hayward, Calif.).

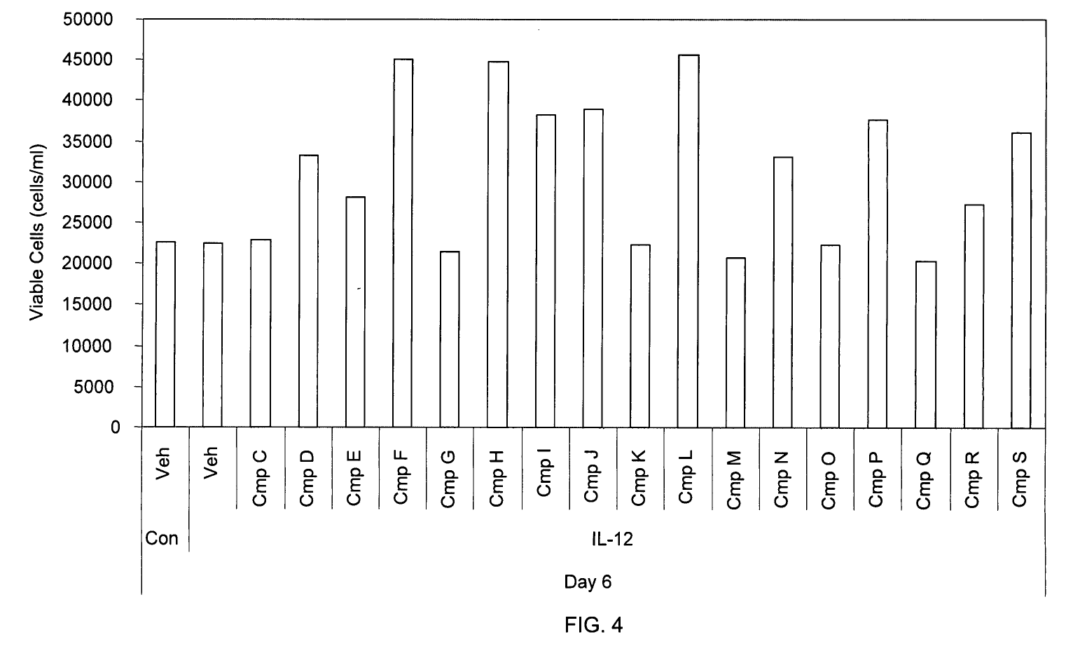

As shown in FIG. 4, compounds of the present invention did not decrease the number of viable T helper cells present at day 6 in these studies. These results demonstrated that methods and compounds of the present invention do not decrease T helper cell number or compromise viability.

### Example 3

### Decreased Interleukin-12-Induced Gene Expression in T Helper Cells

The effect of compounds and methods of the present invention on IL-12-induced gene expression in T helper cells was evaluated as follows. For these studies, analyses were performed to examine the effect of compounds of the present invention on expression of three genes induced by IL-12 in T helper cells: IL12Rβ2, IL18R1, and IL18RAP. (See, e.g., Saremeva et al. (2000) J hnmunol 165:1933-1938; Rogge et al. (1997) J Exp Med 185:825-831; and Nakahira et al. (2001) J Inmunol 167:1306-1312.)

Human cord blood na\dot{i}ve T helper cells (AllCells LLC, Emeryville, Calif.) were stimulated (i.e., T cell receptor (TCR) stimulation) by seeding into 96-well culture dishes coated with 5 μg/ml anti-CD3 antibody (clone UCHT1, R&D Systems, Minneapolis, Minn.), and 5 μg/ml anti-CD28 antibody (clone ANC28.1/D10, Ancell Corporation, Bayport, Minn.), and cultured at 37° C., 5% CO₂ in RPMI-1640 (Mediatech Inc., Herndon, Va.) containing 1% FBS, 1 U/ml penicillin-1 μg/ml streptomycin (Invitrogen Corporation, Carlsbad, California), and 50 U/ml IL-2 (Roche Diagnostics Corporation, Indianapolis, Ind.). At time of seeding, cells were cultured with media alone or with media containing 2 ng/ml IL-12 (R&D Systems, Minneapolis, Minn.) (to stimulate differentiation of the T helper cells into Th1 cells) and 10 μg/ml anti-IL-4 antibody (R&D Systems, Minneapolis, Minn.) (to block differentiation of the T helper cells into Th2 cells). Compound B or 1% DMSO (Veh) was added to the existing culture media and the cells cultured for an additional six days.

The cells were then lysed using Cell-to-Signal lysis buffer (Applied Biosystems, Foster City, Calif.) according to the manufacturer's instructions. The cell lysates were diluted with RNAse-free and DNAse-free water and used as templates in quantitative one-step duplex RT-PCR reactions using QuantiTect Multiplex RT-PCR Kit reagents (Qiagen Inc., Valencia, Calif.) and Taqman primers and probes (Applied Biosystems, Foster City, Calif.) as follows: IL18RAP Taqman primer/probe, catalog no. HS00187256M1; IL-12Rβ2 Taqman primer/probe, catalog no. HS00155486M1; EL18R1 Taqman primer/probe, catalog no. HS00175381M1; and TBET Taqman primer/probe, catalog no. HS00203436M1. RT-PCR reactions were performed according to the manufacturers' instructions. Changes in gene expression of IL12Rβ2, IL18R1, and IL18RAP following IL-12 addition in each sample were normalized relative to 18s ribosomal RNA gene expression measured in the same sample.

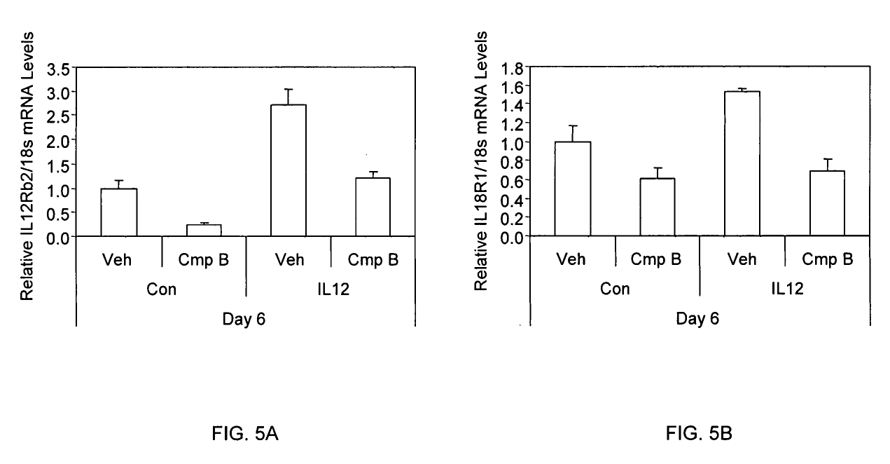

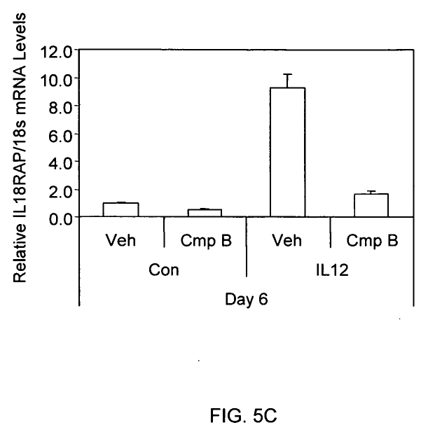

As shown in FIGS. 5A, 5B, and 5C, addition of Compound B to TCR-stimulated T helper cells in the absence of IL-12 resulted in a decrease in gene expression of IL12Rβ2, IL18R1, and IL18RAP, respectively. In TCR-stimulated T helper cells treated with IL-12 in the absence of Compound B, gene expression, as measured by mRNA analysis, of IL12Rβ2, IL18R1, and IL18RAP increased. When Compound B was added to the TCR-stimulated T helper cells in the presence of IL-12, the IL-12-mediated increase in IL12Rβ2, IL18R1, and IL18RAP gene expression was reduced. These results showed that methods and compounds of the present invention are effective at reducing IL-12-mediated increases in expression of these three IL12-responsive genes in T helper cells. Taken together, these results suggested that methods and compounds of the present invention are useful for reducing IL-12-mediated gene expression in activated T helper cells.

### Example 4

### Decreased Cytokine Synthesis by T Helper Cells

To examine the effect of compounds and methods of the present invention on T helper cell synthesis of pro-inflammatory and anti-inflammatory cytokines, the following studies were performed. Human cord blood na\dot{i}ve CD4+T Cells (AllCells LLC, Emeryville, Calif.) were stimulated (i.e., T cell receptor (TCR) stimulation) by seeding into 96-well culture dishes coated with 5 μg/ml anti-CD3 antibody (clone UCHT1, R&D Systems, Minneapolis, Minn.), and 5 μg/ml anti-CD28 antibody (clone ANC28.1/D10, Ancell Corporation, Bayport, Minn.), and cultured at 37° C., 5% CO₂ in RPMI-1640 (Mediatech Inc., Hemdon, Va.) containing 1% FBS, 1 U/ml penicillin-1 μg/ml streptomycin (Invitrogen Corporation, Carlsbad, Calif.), and 50 U/ml IL-2 (Roche Diagnostics Corporation, Indianapolis, Ind.). At time of seeding, cells were treated according to one of the following conditions: 1) media alone (Con); 2) 10 ng/ml IL4, 10 μg/ml anti-IFN-γ antibody, and 10 μg/ml anti-IL-12 antibody (R&D Systems, Minneapolis, Minn.); or 3) 2 ng/ml IL-12 and 10 μg/ml anti-IL-4 antibody. Compound A or 1% DMSO (Veh) was added to existing culture media and the cells were cultured for an additional seven days.

The cell-conditioned media was collected and analyzed for IFN-γ levels (a pro-inflammatory cytokine) and IL-5 levels (an anti-inflammatory cytokine) using MS2400 Human Tissue Culture Kits (MesoScale Technologies, Gaithersburg, Md.) according to the manufacturer's instructions.

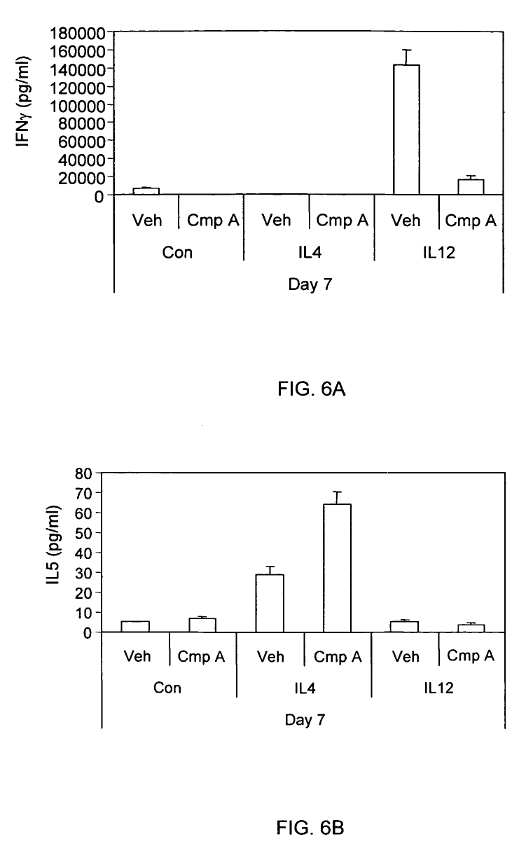

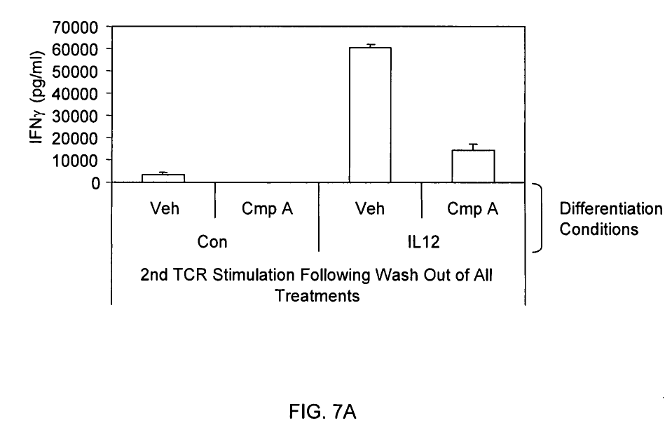

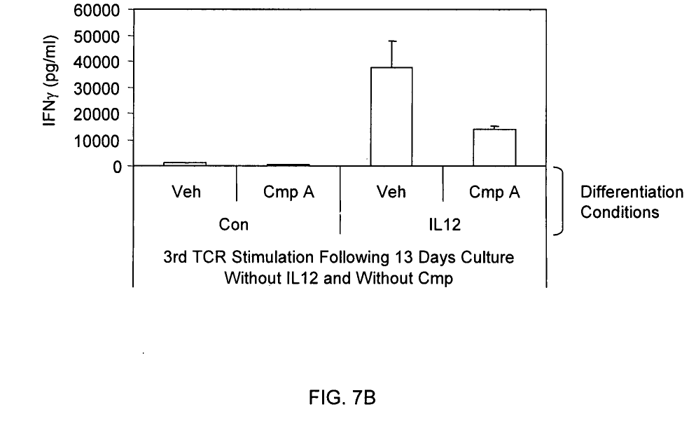

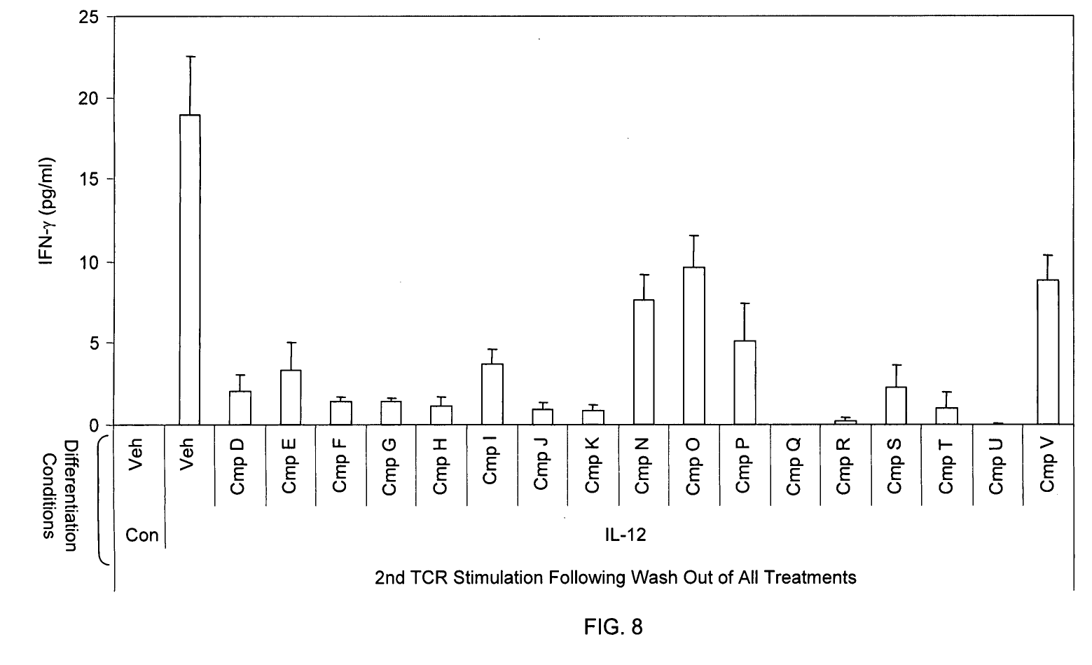

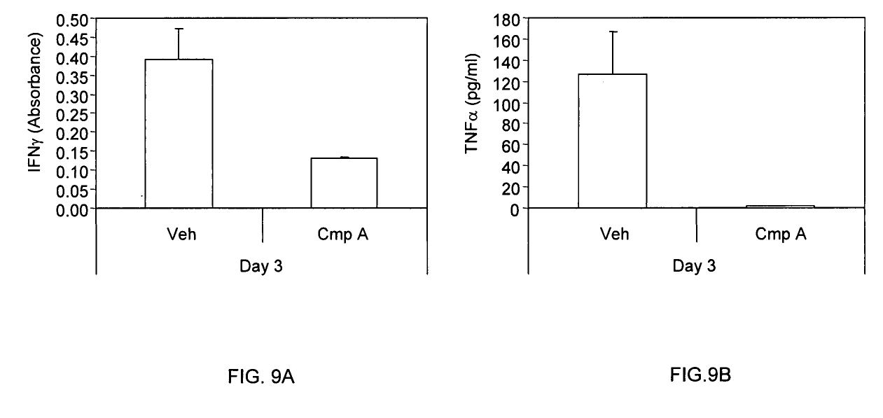

As shown in FIG. 6A, the addition of IL-12 (condition 3 above) to TCR-stimulated T helper cells increased IFN-γ secretion compared to the secretion observed in vehicle-treated control T helper cells, indicating that the TCR-stimulated T helper cells differentiated into Th1 cells in response to IL-12. As expected, IL-4 had no effect on lFN-γ secretion from these cells. However, when TCR-stimulated T helper cells were treated with IL-12 in the presence of Compound A, the IL-12-mediated increase in IFN-γ secretion was not observed. The addition of Compound A in the presence of IL-12 thus reduced the IL-12-mediated increase in IFN-γ secretion. These results showed that compounds and methods of the present invention are effective at reducing IL-12-mediated increases in IFN-γ secretion from activated T helper cells.

As shown in FIG. 6B, the addition of IL-4 (condition 2 above) to TCR-stimulated T helper cells increased IL-5 secretion compared to the secretion observed in vehicle-treated control T helper cells, indicative of the TCR-stimulated T helper cells having differentiated into Th2 cells in response to IL-4 stimulation. As expected, IL-12 had no effect on IL-5 secretion from these cells. Addition of Compound A to TCR-stimulated T helper cells treated with IL-4 resulted in increased IL-5 levels compared to that observed in TCR-stimulated T helper cells treated with IL-4 alone. These results indicated that methods and compounds of the present.invention inhibited IL-12 signaling in T helper cells, but do not block IL-4 signaling in T helper cells.

Taken together, these results showed that methods and compounds of the present invention are effective at decreasing secretion of pro-inflammatory cytokines from T helper cells (i.e., T helper cells that have differentiated into Th1 cells) while not effecting secretion of anti-inflammatory cytokines from T helper cells (i.e., T helper cells that have differentiated into Th2 cells).

### Example 5

### Inhibition of Th1 Cell Differentiation

The effectiveness of compounds of the present invention at reducing Th1 differentiation was examined as follows. Th1 differentiation was induced by culturing anti-CD3 stimulated T cells in the presence of exogenous IL-12. Human cord blood na\dot{i}ve T helper cells (AllCells LLC, Emeryville, Calif.) were stimulated (i.e., T cell receptor (TCR) stimulation) by seeding into 96-well culture dishes coated with 5 μg/ml anti-CD3 antibody (clone UCHT1, R&D Systems, Minneapolis, Minn.), and 5 μg/ml anti-CD28 antibody (clone ANC28.1/D10, Ancell Corporation, Bayport, Minn.), and cultured at 37° C., 5% CO₂ in RPMI-1640 (Mediatech Inc., Hemdon, Va.) containing 1% FBS, 1 U/ml penicillin-1 μg/ml streptomycin (Invitrogen Corporation, Carlsbad, Calif.), and 50 U/ml IL-2 (Roche Diagnostics Corporation, Indianapolis, Ind.). At time of seeding, cells were cultured with media alone or with media containing 2 ng/ml IL-12 and 10 μg/ml anti-IL-4 to induce Th1 cell differentiation. Compound A or 1% DMSO (Veh) was added to existing culture media and the cells cultured for an additional seven days. These steps lead to a first T cell receptor stimulation of the T helper cells.

The cells were then collected, washed twice with RPMI-1640 media containing 1% FBS, and counted before equal numbers of cells per well were re-stimulated (second T cell receptor (TCR) stimulation following washout of all prior treatments) by re-seeding the cells into 96-well culture dishes coated with 5 μg/ml anti-CD3 antibody and 5 μg/ml anti-CD28 antibody. The cells were then cultured at 37° C., 5% CO₂ in RPMI-1640 media containing 1% FBS, 1 U/ml penicillin-1 μg/ml streptomycin, and 50 U/ml IL-2. After 24 hours the conditioned media from the cells was collected and analyzed for IFN-γ levels using a MS2400 Tissue Culture Kit (MesoScale Technologies, Gaithersburg, Md.) according to the manufacturer's instructions.

As shown in FIG. 7A, as compared to T helper cells activated by TCR stimulation alone (i.e., TCR stimulation of the T helper cells by plating on anti-CD3 antibody and on anti-CD28 antibody) in the absence of IL-12, TCR-stimulated T helper cells stimulated in the presence of IL-12 (to induce the differentiation of the T helper cells into Th1 cells) secreted higher levels of IFN-γ following a second TCR stimulation in the absence of IL-12. (See IL-12 differentiation conditions, Veh bar in FIG. 7A.) The presence of Compound A during the first TCR-stimulation in the presence of IL-12 inhibited IL-12-induced T cell differentiation as evidenced by reduced IFN-γ secretion following the second TCR stimulation. (See IL-12 differentiation conditions, Cmp A bar in FIG. 7A.) These results showed that Compound A inhibited IL-12-induced Th1 differentiation, as no compound was present during the second TCR stimulation yet the cells showed decreased IFN-γ secretion.

Thirteen days after the second TCR stimulation described above, the cells were collected, washed twice with RPMI-1640 media containing 1% FBS, and counted before equal numbers of cells per well were stimulated a third time (third TCR stimulation) by re-seeding into 96-well culture dishes coated with 5 μg/ml anti-CD3 antibody and 5 μg/ml anti-CD28 antibody. The cells were then cultured at 37° C., 5% CO₂ in RPMI-1640 containing 1% FBS and 1 U/ml penicillin-1 μg/ml streptomycin. After 24 hours the conditioned media was collected and analyzed for IFN-γ levels using a MS2400 Tissue Culture Kit (MesoScale Technologies, Gaithersburg, Md.) according to the manufacturer's instructions.

As shown in FIG. 7B, the inhibition of IL-12 induced Th1 differentiation by Compound A was sustained. Specifically, TCR-stimulated T helper cells that were differentiated with IL-12 in the absence or presence of Compound A maintained a polarized Th1 cell or na\dot{i}ve T cell phenotype, respectively, as evidenced by decreased IFN-γ secretion after a third TCR stimulation. (Compare IL-12 differentiation conditions, Veh bar in FIG. 7B with IL-12 differentiation conditions, Cmp A bar in FIG. 7B.) These results showed that Compound A inhibited IL-12-induced Th1 differentiation, as no compound was present during the second and third TCR stimulation but reduced IFN-γ secretion was observed.

In another series of experiments, the effectiveness of compounds of the present invention at reducing Th1 differentiation was determined. Human cord blood na\dot{i}ve T helper cells were subjected to a first and a second TCR stimulation as described above with the following modifications: cell cultures for the first and second TCR stimulation contained 100 U/ml penicillin-100 U/ml streptomycin and the first TCR stimulation was carried out for six days. Following the second TCR stimulation, conditioned media from the cells was collected and analyzed for IFN-γ levels using a MS2400 Tissue Culture Kit (MesoScale Technologies, Gaithersburg, Md.) according to the manufacturer's instructions.

As shown in FIG. 8, as compared to T helper cells activated by TCR stimulation alone (i.e., TCR stimulation of the T helper cells by plating on anti-CD3 antibody and on anti-CD28 antibody) in the absence of IL-1 2, TCR-stimulated T helper cells stimulated in the presence of IL-12 (to induce the differentiation of the T helper cells into Th1 cells) secreted higher levels of IFN-γ following a second TCR stimulation in the absence of IL-12. (See IL-12 differentiation conditions, Veh bar in FIG. 8.) The presence of compounds of the present invention during the first TCR-stimulation in the presence of IL-12 inhibited IL-12-induced T cell differentiation as evidenced by reduced IFN-γ secretion following the second TCR stimulation. (See, e.g., IL-12 differentiation conditions, Cmp D bar in FIG. 8.) These results showed that compounds of the present invention inhibited IL-12-induced Th1 differentiation, as no compound was present during the second TCR stimulation yet the cells showed decreased IFN-γ secretion.

Taken together, results from these series of experiments showed that compounds and methods of the present invention are effective at inhibiting Th1 cell differentiation as measured by IFN-γ secretion following TCR stimulation.

### Example 6

### Decreased IFN-γ and TNF-α Levels Ex Vivo Following T Cell Activation In Vivo

The effect of methods and compounds of the present invention on INF-γ and TNF-α expression following in vivo activation was examined as follows. In these experiments, in vivo T cell activation was first induced by eliciting an inflammatory reaction in rats by injection of peptidoglycan-polysaccharide polymers. An exemplary compound of the present invention was then administered to the animals. T cells were then isolated from the blood of the animals and analyzed ex vivo for IFN-γ and TNF-α secretion.

Female Lewis rats (150-200 gm; Harlan, Indianapolis, Ind.) were injected intra-peritoneal with peptidoglycan-polysaccharide polymers (15 μg/gm body weight, Lee Laboratories, Grayson, Ga.). Animals were administered Compound A (40 mg/kg) via oral gavage on days 28, 30, 32, 35, 37, 39, 42, and 44 following peptidoglycan-polysaccharide polymer injection. Peripheral blood mononuclear cells (PMBCs) were isolated from the blood of the animals by Ficol gradient (BD Vacutainer CPT tubes) on day 44.

Equal numbers of PMBCs (50,000 cells/well) were plated on culture dishes coated with anti-CD3 antibodies in RPMI-1640 media containing 10% serum, and the cells were cultured for 72 h. Supernatants from the cell cultures were tested for IFN-γ and TNF-α levels using commercially-available ELISA assays (R&D).

As shown in FIGS. 9A and 9B, in vivo administration of Compound A to animals undergoing an inflammatory reaction, in which T cells are activated, was effective at decreasing both IFN-γ and TNF-α secretion from the activated T cells, respectively, as measured ex vivo. These results indicated that the methods and compounds of the present invention are effective at inhibiting the differentiation of T helper cells into Th1 cells in vivo.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for inhibiting the differentiation of a T helper cell into a Th1 cell, the method comprising contacting the T helper cell with an effective amount of a compound that inhibits the activity of a HIF prolyl hydroxylase enzyme, thereby inhibiting the differentiation of the T helper cell into a Th1 cell.

2. A method for inhibiting the differentiation of a T helper cell into a Th1 cell in a subject, the method comprising administering to the subject an effective amount of a compound that inhibits the activity of a HIF prolyl hydroxylase enzyme, thereby inhibiting the differentiation of the T helper cell into a Th1 cell in the subject.

3. A method for inhibiting IL-12 signaling in a T helper cell, the method comprising contacting the T helper cell with an effective amount of a compound that inhibits the activity of a HIF prolyl hydroxylase enzyme, thereby inhibiting IL-12 signaling in the T helper cell.

4. A method for inhibiting IFN-γ secretion from a T helper cell, the method comprising contacting the T helper cell with an effective amount of a compound that inhibits the activity of a HIF prolyl hydroxylase enzyme, thereby inhibiting the IFN-γ secretion from the T helper cell.

5. A method for inhibiting TNF-α secretion from a T helper cell, the method comprising contacting the T helper cell with an effective amount of a compound that inhibits the activity of a HIF prolyl hydroxylase enzyme, thereby inhibiting the TNF-α secretion from the T helper cell.
